(19) United States
(12) Patent Application Publication
(10) Pub. No.: US 2011/0257527 A1
(43) Pub. Date: Oct. 20, 2011

(54) ULTRASOUND CAROTID MEDIA WALL CLASSIFICATION AND IMT MEASUREMENT IN CURVED VESSELS USING RECURSIVE REFINEMENT AND VALIDATION

(76) Inventor: Jasjit S. Suri, Roseville, CA (US)

(21) Appl. No.: 13/077,631

(22) Filed: Mar. 31, 2011

Related U.S. Application Data
(63) Continuation-in-part of application No. 12/799,177, filed on Apr. 20, 2010.
Application No. 12/799,177 is a continuation-in-part of application No. 12/802,431, filed on Jun. 7, 2010.
Application No. 12/802,431 is a continuation-in-part of application No. 12/896,875, filed on Oct. 2, 2010.
Application No. 12/896,875 is a continuation-in-part of application No. 12/960,491, filed on Dec. 4, 2010.
Application No. 12/960,491 is a continuation-in-part of application No. 13/053,971, filed on Mar. 22, 2011.

(51) Int. Cl. A61B 8/00

(52) U.S. Cl. 600/440

(57) ABSTRACT

A computer-implemented system and method for intima-media thickness (IMT) measurements using a validation embedded segmentation method. Various embodiments include receiving biomedical imaging data and patient demographic data corresponding to a current scan of a patient; checking the biomedical imaging data in real-time to determine if an artery of the patient has a calcium deposit in a proximal wall of the artery; acquiring arterial data of the patient as a combination of longitudinal B-mode and transverse B-mode data; using a data processor to automatically recognize the artery by embedding anatomic information; using the data processor to calibrate a region of interest around the automatically recognized artery; automatically computing the weak or missing edges of intima-media and media-adventitia walls using labeling and connectivity; and determining the intima-media thickness (IMT) of an arterial wall of the automatically recognized artery.

### PRIORITY APPLICATION

This is a continuation-in-part patent application of co-pending patent application Ser. No. 12/799,177; filed Apr. 20, 2010 by the same applicant. This is also a continuation-in-part patent application of co-pending patent application Ser. No. 12/802,431; filed Jun. 7, 2010 by the same applicant. This is also a continuation-in-part patent application of co-pending patent application Ser. No. 12/896,875; filed Oct. 2, 2010 by the same applicant. This is also a continuation-in-part patent application of co-pending patent application Ser. No. 12/960,491; filed Dec. 4, 2010 by the same applicant. This is also a continuation-in-part patent application of co-pending patent application, Ser. No. 13/053,971 (title: IMAGING BASED SYMPTOMATIC CLASSIFICATION AND CARDIOVASCULAR STROKE RISK SCORE ESTIMATION); filed Mar. 22, 2011 by the same applicant. This present patent application draws priority from the referenced co-pending patent applications. The entire disclosures of the referenced co-pending patent applications are considered part of the disclosure of the present application and are hereby incorporated by reference herein in its entirety.

### TECHNICAL FIELD

This patent application relates to methods and systems for use with data processing, data storage, and imaging systems, according to one embodiment, and more specifically, for ultrasound image processing.

### BACKGROUND

### 1. Introduction

The state of Atherosclerosis in carotids or other blood vessels can be studied using MRI or Ultrasound. Because ultrasound offers several advantages like real time scanning of carotids, compact in size, low cost, easy to transport (portability), easy availability and visualization of the arteries are possible, Atherosclerosis quantification is taking a new dimension using ultrasound. Because one can achieve compound and harmonic imaging which generates high quality images with ultrasound, it is thus possible to do two-dimensional (2D) and three-dimensional (3D) imaging of carotid ultrasound for monitoring of Atherosclerosis.

In recent years, the possibility of adopting a composite thickness of the tunica intima and media, i.e., an intima-media thickness (hereinafter referred to as an “IMT”) of carotid arteries, as surrogate marker for cardiovascular risk and stroke. Conventional methods of imaging a carotid artery using an ultrasound system, and measuring the IMT using an ultrasonic image for the purpose of diagnosis are being developed.

A conventional measuring apparatus can measure an intima-media thickness of a blood vessel using an ultrasound device to scan the blood vessel. Then, for example, an image of a section of the blood vessel including sections of the intima, media and adventitia is obtained. The ultrasound device further produces digital image data representing, this image, and outputs the digital image data to a data analyzing device.

The intima, media and adventitia can be discriminated on the basis of changes in density of tissue thereof. A change in density of tissue of the blood vessel appears as a change of luminance values in the digital image data. The data analyzing device detects and calculates the intima-media thickness on the basis of the changes of luminance values in the digital image data. The digital image data can include a plurality of luminance values each corresponding to respective one of a plurality of pixels of the image. The data analyzing device can set a base position between a center of the blood vessel and a position in a vicinity of an inner intimal wall of the blood vessel on the image, on the basis of a moving average of the luminance values. The data analyzing device can detect a maximum value and a minimum value from among the luminance values respectively corresponding to a predetermined number of the pixels arranged from the base position toward a position of an outer adventitial wall on the image. The data analyzing device can then calculate the intima-media thickness on the basis of the maximum value and the minimum value.

The major challenges which can be affected in finding the IMT are: (a) how well the ultrasound probe is gripped with the neck of a patient to scan the carotids; (b) how well the ultrasound gel is being applied; (c) the orientation of the probe; (d) demographics of the patient; (e) skills of the sonographer or vascular surgeon; (f) gaps in the intensity distribution along the adventitia walls of the carotid ultrasound images; (g) shadows cones in the adventitia borders due the presence of calcium deposits; (h) threshold chosen for finding the peaks corresponding to the LI and MA points for each signal orthogonal to the lumen; (i) variability in the lumen region; (j) variability in the geometric shapes of the carotid scans such as convex, concave, up-hill, down-hill, and finally, (k) handing the large databases to process large number of images.

Thus, a system and method for fast, reliable and automated method for IMT measurements is needed.

### BRIEF DESCRIPTION OF THE DRAWINGS

FIG. 1 shows the IMT measurement system.

FIG. 2 is the overall AtheroEdge™ system.

FIG. 3 shows the image reduction processor based on wavelet transform or pyramid filter, which can down sample the image.

FIG. 4 shows the process for despecking filtering. The process uses the scaling of the original pixel. The noise variance process is being used by the scale processor.

FIG. 5 is the filter processor.

FIG. 6 shows the computation of the noise variance processor.

FIG. 7 shows the validation processor.

FIG. 8 shows the recognition (stage I) and calibration processor (stage II).

FIG. 9 is the details of the artery validation processor.

FIG. 10 is the lumen identification system.

FIG. 11 shows the Lumen Classifier Processor for segmentation of Lumen region in the longitudinal carotid image.

FIG. 12 shows the Calibration Processor (stage II). This consists of the recursive classification stage using means shift, LI/MA reconstruction processor and LI/MA smoothing processor.

FIG. 13: show the reconstruction process example. FIG. 14 (A) is the ADF profile from the output of stage-I. FIG. 14 (B) signal along the profile P1-P2. FIG. 14 (C) is the binary image showing the media wall. FIG. 14 (D) for reconstruction of the MA point along the profile b in FIG. 14 (C). FIG. 14 (E) is the longest MA trend reconstructed. FIG. 14 (F) is the final MA reconstructed border.

FIG. 14: show the original images cropped with the smallest rectangle possible including the entire Guidance Zone (obtained starting from the AD_{F} profile and extending it upwards 50 pixels).

FIG. 15 shows the images obtained by creating a color label image of the output image from the MSC algorithm and overlaying them on the original cropped images.

FIG. 16 shows the output images from the mean shift classifier (MSC) algorithm. The images are presented as black and white segmentation masks.

FIG. 17 shows the binary images in which the objects correspond to the pixels which are found to belong to the IM_{class}.

FIG. 18 shows the original cropped images that have the longest trend of the preliminary MA profile overlaid in white (first CAILRS_{MA}).

FIG. 19 show the original cropped images that have the final MA profile overlaid in white (CAILRS_{MA}).

FIG. 20 shows the original cropped images that have the longest trend of the preliminary LI profile overlaid in white (first CAILRS_{LI}).

FIG. 21 shows the original cropped images that have the final LI profile overlaid in white (CAILRS_{LI}).

FIG. 22 shows the original cropped images that have the final MA and LI profiles overlaid in white (CAILRS_{MA} and CAILRS_{LI}, respectively).

FIG. 23 shows the comparison between CAILRS and expert tracings on two different images. (A, C) Gray-scale images shown with computed LI border in a continuous white line and Ground Truth LI border in a dashed white line. (B, D) Gray-scale images shown with computed MA border in a continuous black line and Ground Truth MA border in a dashed black line.

FIG. 24 shows the comparison of Automated CAILRS and semi-automated FOAM for LI and MA borders. (A, D) Original gray-scale images automatically cropped in Stage-I. (B, Gray-scale images shown with computed CAILRS borders overlaid in white. (C, F) Gray-scale images shown with computed semi-automated FOAM borders overlaid in white.

FIG. 25 shows the correlation plot of CAILRS and FOAM IMT measurements versus manual tracings by experts.

FIG. 26 Bland-Altman plot of CAILRS and FOAM IMT biases.

FIG. 27 shows the AtheroEdge™ parameters and experimental values used.

FIG. 28 shows the parameters used in stage II (Calibration stage) using mean shift classifier and reconstruction of LI/MA borders and LI/MA refinement.

FIG. 29 shows the comparative performance evaluation of CAILRS, semi-automated FOAM and automated methods: CALEX, CULEX, CALSFOAM, and CAUDLES.

FIG. 30 is a processing flow diagram illustrating an example embodiment of a computer-implemented system and method for fast, reliable, and automated embodiments for using a validation embedded multi-resolution edge flow approach to vascular ultrasound for intima-media thickness (IMT) measurement as described herein.

FIG. 31 shows a diagrammatic representation of machine in the example form of a computer system within which a set of instructions when executed may cause the machine to perform any one or more of the methodologies discussed herein.

### DETAILED DESCRIPTION

Recognition of the carotid artery consists of finding a regional layer close to the carotid artery and possibly all along the carotid artery in the image frame. This recognition process must ensure that we are able to distinguish the carotid artery layer from other veins such as jugular vein (JV). We modeled the carotid artery recognition process by taking the hypothesis that carotid artery's far wall adventitia is the brightest in the ultrasound scan frame; hence if we can automatically find this layer, then segmentation process of the far wall would be more systematic and channeled. Since the scanning process of carotid artery yields varying geometries of the carotid artery in the ultrasound scans, one has to ensure that the recognition process is able to handle various geometric shapes of the carotid arteries in the images. The process of location of far adventitia bright layer in the image frame can be supported by the fact that it is very close to lumen region, which carries the blood to the brain. Taking these two properties of the carotid artery ultrasound scan, this patent application has modeled the recognition process as a tubular model where the walls are considered as bright layers of the scan which can be picked up by the high intensity edge detector. Our edge model must keep in mind that the far adventitia layers are about a millimeter thick (which is about 16 pixels in image frame). Thus one would need to find an edge operator (preferably Gaussian in nature) which has an ability to have a width (scale) region of as wide as 8 pixels in the image frame. We have modeled this width to be the scale factor of the Gaussian kernel, where the scale is the standard deviation of the edge operator. The ability of finding this edge can be obtained by convolving the image region with a derivative of the Gaussian Kernel having a scale factor as rationalized in the edge model. Thus the whole idea of finding automatically the far adventitia border can be brought in the frame work of scale-space, where the image is convolved with first or higher order derivatives of Gaussian Kernel with known scale (s). While the scale-space model is fancy in itself, one must remember that it is very important to have the scale nearly fitting the far adventitia border region. Since the image frame is large enough to have a wider scale, we therefore have further adapted an approach where the scale-space model will behave consistent with respect to the image size. This requires that image be down sampled to half before the scale-space model can be adapted. Thus one can call this framework to be more like a multi-resolution thereby using the correct scale for capturing the edges of the far adventitia layers. Thus our architecture for stage I is the recognition of the far adventitia location in the grayscale image of the carotid artery using multi-resolution approach in scale-space framework.

In the following description, for purposes of explanation, numerous specific details are set forth in order to provide a thorough understanding of the various embodiments. It will be evident, however, to one of ordinary skill in the art that the various embodiments may be practiced without these specific details.

This patent application discloses various embodiments of a computer-implemented system and method for fast, reliable, and automated embodiments for vascular ultrasound for validation embedded LIMA segmentation and intima-media thickness (IMT) measurement. In particular, this patent application discloses various embodiments of a computer-implemented system and method for intima-media thickness (IMT) measurements using (a) a validation embedded segmentation method in stage I, (b) recursive classification, (c) LI/MA reconstruction and (d) LI/MA refinement. The various embodiments described herein also include the features described in more detail below.

Coarse to Fine Resolution Processing: Previous art has focused on methods for either classification of media layer or finding the MA edges in the manual designated Region of Interest (ROI). Since it is manual ROI, it is time consuming and non-practical for clinical applications, we have developed a new method which is fast, accurate, reliable and very practical for IMT measurement for carotids, brachial, femoral and aortic blood vessels. Since the manual methods are time consuming and requires a lot of training, this applications is a two step stage process: (a) automated validation embedded artery recognition and (b) automated calibration using (i) recursive classification, (ii) LI/MA reconstruction and (iii) LI/MA refinement. The automated recognition process is challenging given the Jugular vein in the neighborhood. Our concept is to recognize the artery in a smaller image with a high speed (so-called coarse resolution) and recognize the artery. The spotted artery can then be seen in the fine resolution or high resolution. This will allow processing the pixels in the correct region of interest. The statistics of the neighboring pixels will not affect the region of interest, which is where the accurate LIMA borders need to be determined. Normally, arteries are about 10 mm wide while the media thickness is about 1 mm wide. It is also known from our experience that the image resolution is about 15-17 pixel per mm. If we can bring the original resolution to a coarse resolution by one step down sample, we can bring the media layer to about 8 pixels per mm. Further, if this coarse resolution is down sampled by another half, then one can bring the image resolution from 8 pixels/mm to 4 pixels/mm. Thus, if the coarse resolution of the arterial ultrasound vessels has a medial thickness of 4 pixels/mm, one can easily detect such edges by convolving the higher order derivatives of Gaussian kernel with the coarse resolution image. Thus, a new concept here (in stage I) is to automatically detect the arterial wall edges by down sampling the image and convolving the coarse images to higher order derivatives of Gaussian kernels. This allows the media layer to be automatically determined. Such an approach for automated media layer detection from fine to coarse resolution will further improve the region of interest determination. The art of changing the fine to coarse resolution has been popular in computer vision sciences. There are several methods available to converting the image from high resolution to coarse resolution. One of them is wavelet-based method where wavelets are being applied for down sampling the image to half. Another method can be hierarchical down sampling method using Peter Burt's algorithm. Thus the first advantage of the current system is automated recognition of the artery at coarse resolution and then using the MA border for visualization and recognition at the fine resolution (up-sampled resolution). This scheme has several advantages to it:
(1) Robustness and Accurate Wall Capture: it is very robust because the higher order derivative kernels are very good in capturing the vessel walls (see, A Review on MR Vascular Image Processing Algorithms: Acquisition and Pre-filtering: Part I, Suri et al., IEEE TRANSACTIONS ON INFORMATION TECHNOLOGY IN BIOMEDICINE, VOL. 6, NO. 4, pp. 324-337, DECEMBER 2002; and A Review on MR Vascular Image Processing: Skeleton Versus Nonskeleton Approaches: Part II, Suri et al., IEEE TRANSACTIONS ON INFORMATION TECHNOLOGY IN BIOMEDICINE, VOL. 6, NO. 4, DECEMBER 2002).
(2) Scale-space Method for Automated Recognition: Since the scale of the derivative Gaussian Kernels are determined using the anatomic information of the artery along with the multi-resolution framework, the system is robust to find the far adventitia borders.
(3) Validation Embedded Segmentation of Vascular IMT estimation: Here the recognition of artery has been validated by the anatomic information during the segmentation process. Since lumen is the anatomic information which is blood carrier to brain and is next to the far adventitia borders, which needs to be located, therefore, this patent application uses the anatomic information (lumen) to ensure that the far adventitia borders are robustly computed along the CCA/ICA, and do not penetrate the lumen region or near wall region. This adds robustness to our automated recognition and IMT measurement system.
(4) Faster than the conventional processing: Since the recognition is strategized at coarse level down sampled twice from its original size of the image, it is therefore processing ¼^{th} the number of pixels for automated recognition of the media layer. This improves the speed of the system.
(5) Independent of Orientation of the vascular scan: Another major advantage to the system is that these Gaussian kernels are independent of the orientation of the blood vessels in the image. Since the ultrasound vascular scans do not always have the vessel orientation horizontal with respect bottom edge of the image, manual methods can pose a further challenge towards the region of interest estimation.
(6) Guiding Method for the Calibration System: Since the recognition is followed by the calibration process, the calibration system becomes very robust since the calibration processing is done in the region of interest determined by the automated validation embedded recognition system. Thus the calibration system adds the value determined by the automated recognition system for vascular ultrasound such as IMT measurement for carotid, femoral, aortic and brachial. Such a combination where the calibration system is guided by the automated recognition system helps in mass processing of huge database processing.
(7) Running the Mass IMT system for Clinical Analysis: Since the recognition is automated followed by the calibration system, the largest value such a system would deliver will be in its real time use for analysis of IMT measurement on a large databases. Running clinical databases on still images would be even more beneficial because such a system would be completely automated in terms of arterial recognition and IMT measurement.
(8) Applications: Since the ultrasound probes use almost the same frequency of operation for scanning the vascular arteries such as carotid, femoral, brachial and aortic, it is thus possible to use such a system for these blood vessels.

In the prior art, we have seen that the speckle reduction has been used for removing speckles in the ultrasound images. Though speckle reduction is common in ultrasound imaging, but the way speckle reduction is used here is very conservative. The idea here is to find out where the LIMA borders are using automated recognition system and then apply the local statistical speckle reduction filter in specific set of pixels which come under the LIMA band or media layer. Such a strategy allows multiple advantages:
(1) Avoiding Large Computation Times on Speckle Reduction: The computation time for speckle reduction is not wasted in such a strategy, unlike conventional methods, where the speckle reduction is part of the whole streamline flow and is being run on the whole image.
(2) Speckle Reduction is implemented on the original raw intensity in the region estimated at a Coarse Resolution: Second, the speckle reduction filter is run in the automated recognized region (MA borders) which is actually applied on the original image rather than on the coarse image. This way the original speckles are removed preserving the intensities of high gradient structures like LI and MA peaks. This is very important because the calibration system acts on these speckle reduction region of interest.
(3) Guidance to the Calibration System: The calibration system is guided by the speckle reduction filter which is optimized for the region of interest.

Extracting LIMA borders in presence of Calcium Shadow: Calcium is an important component of the media layer. It is not exactly known how the calcium is formed, but it is said that calcium accumulates in the plaques. During the beginning of Atherosclerosis disease, the arterial wall creates a chemical signal that causes a certain type of WBC (white blood cells) such as monocytes and T cells that attaches the arterial wall. These cells then move into the wall of the artery. These T cells or monocyles are then transformed into foam cells, which collect cholesterol and other fatty materials and trigger the growth of the muscle cells (which are smooth in nature) in the artery. Over time, it is these fat-laden foam cells that accumulate into plaque covered with a fibrous cap. Over time, the calcium accumulates in the plaque. Often times, the calcium is seen in the near wall (proximal wall) of the carotid artery or aortic arteries. This causes the shadow cone formation in the distal wall (far wall). As a result the LI boundaries are over computed from its actual layer. The shadow causes the LI lining over the actual LI boundary. As a result, the LI-MA distances are over computed in the shadow zone. Because of this, the IMT formation is over computed in these cases.

This application particularly takes care of IMT computation during the shadow cone formation. We will see how the actual LI boundaries are recovered if calcium is present causing the shadow cone. As a result, the IMT computation has the following advantages when using shadow cones.
(1) Accurate IMT computation in real time when the calcium is present in the proximal wall (near wall) causing the shadow cone formation.
(2) The system allows computing the IMT in both cases: (a) when calcium is present and when calcium is not present.

### BRIEF SUMMARY OF AN EXAMPLE EMBODIMENT

The completely automated technique we developed and named CAILRS (class of AtheroEdge™ systems) consists of two steps: (i) the automated validation embedded recognition of the CA in the image frame, and (ii) the segmentation of the far carotid artery wall

using (i) recursive classification, (ii) LI/MA reconstruction and (iii) LI/MA refinement. The output of the stage. II yields LI/MA profiles which is then used for, the IMT measurement.

Cropping System: Preliminarily, the raw ultrasound image is automatically cropped in order to discard the surrounding black frame containing device headers and image/patient data (1). If the image came in DICOM format, we relied on the data contained in the specific field named SequenceOfUltrasoundRegions, which contains four sub-fields that mark the location of the image containing the ultrasound representation. These fields are named RegionLocation (their specific label is xmin, xmax, ymin and ymax) and they mark the horizontal and vertical extension of the image. The raw B-Mode image is then cropped in order to extract only the portion that contains the carotid morphology. Those skilled in the art of DICOM will know that if the image came in from other formats or if the DICOM tags were not fully formatted, one can adopt a gradient-based procedure. We computed the horizontal and vertical Sobel gradient of the image. The gradients repeat similar features for the entire rows/columns without the ultrasound data: they are zero at the beginning and at the end. Hence, the beginning of the image region containing the ultrasound data can be calculated as the first row/column with gradient different from zero. Similarly, the end of the ultrasound region is computed as the last non-zero row/column of the gradient.

Automatic Recognition of the CA: To automatically identify the CA in the image frame, we developed a novel and low-complexity procedure. Following sample steps are used for automatic CA recognition, starting with the automatically cropped image which constitutes the input of the procedure.
Downsampling. The image was first down-sampled by a factor of 2 (i.e., the number of rows and columns of the image was halved).
Speckle reduction. Speckle noise was attenuated by using a first-order statistics filter (named as lsmv by the authors (2, 3)), which gave the best performance in the specific case of carotid imaging. This filter is defined by the following equation:

J_{x,y}=Ī+l_{x,y}(I_{x,y}−Ī) (1)

where, I_{x,y} is the intensity of the noisy pixel, Ī is the mean intensity of a N×M pixel neighborhood and k_{x,y} is a local statistic measure. The noise-free pixel is indicated by J_{x,y}. Loizou et al., (2) mathematically defined

$${k}_{x , y} = \frac{{σ}_{I}^{2}}{{\overline{I}}^{2} {σ}_{I}^{2} + {σ}_{n}^{2}} ,$$

where σₗ² represents the variance of the pixels in the neighborhood, and σₙ² the variance of the noise in the cropped image. An optimal neighborhood size was shown to be 7×7.
Higher order Gaussian derivative filter. The despeckled image was filtered by using a first order derivative of a Gaussian kernel filter. It is possible to observe how the CA walls become enhanced to white. The sigma parameter of the Gaussian derivative kernel was taken equal to 8 pixels, i.e. to the expected dimension of the IMT value. In fact, an average IMT value of say 1 mm corresponds to about 16 pixels in the original image scale and, consequently, to 8 pixels in the down-sampled scale.
AD_{F} refinement using anatomic information and spike removal. The traced AD_{F} profile could be characterized by spikes and false points identification. This could be due to several reasons such as variations in intensities, gaps in the media walls, presence of jugular vein, shadow effects or combination of these. This innovation, therefore introduced a validation protocol, which provides a check on the far adventitia (AD_{F}) profile ensuring that the location of carotid artery is at correct place and the segmentation edge is not very bumpy. This validation step refines the far adventitia (AD_{F}) profile and is done in two steps: (a) refinement using anatomic lumen and (b) spike removal.
   Refinement by anatomic reference (Lumen). This check has been introduced to avoid error conditions of AD_{F} curve protruding into the lumen vessel. Thus, the refinement step requires the identification of the lumen region automatically. We have modeled the lumen segmentation region as a classification process with two classes. Carotid characteristics can be thought of as a mixture model with varying intensity distributions. This is because (a) the pixels belonging to the vessel lumen are characterized by low mean intensity and low standard deviation; (b) pixels belonging to the adventitia layer of the carotid wall are characterized by high mean intensity and low standard deviation; and (c) all remaining pixels should have high mean intensity and high standard deviation. As a result of this distribution, we derived a bi-dimensional histogram (2DH) of the carotid image. For each pixel, we considered a 10×10 neighborhood of which we calculated the mean value and the standard deviation. The mean values and the standard deviations were normalized to 0 and 1 and were grouped into 50 classes each having an interval of 0.02. The 2DH was then a joint representation of the mean value and standard deviation of each pixel neighborhood. It is well established that pixels belonging to the lumen of the artery are usually classified into the first classes of this 2DH: expert sonographer manually traced the boundaries of the CCA lumen and observed the distribution of the lumen pixels on the 2DH. Overall results revealed that pixels of the lumen have a mean values classified in the first 4 classes and a standard deviation in the first 7 classes. We therefore consider a pixel as possibly belonging to the artery lumen if its neighborhood intensity is lower than 0.08 and if its neighborhood standard deviation is lower than 0.14. This method shows how the local statistic is effective in detecting image pixels that can be considered as belonging to the CCA lumen. The AD_{F} points along the CA are considered one by one. For each AD_{F} point:
   1. We consider the sequence of the 30 pixels above it (i.e., the 30 pixels located above the AD_{F} point, towards the top of the image, and, therefore, with lower row indexes).
   2. The AD_{F} point failed the lumen test, if the AD_{F} point crosses the lumen region and has penetrated the lumen region by at least 30 pixels inside the lumen or more. These failed points must not belong to the AD_{F} boundary. These AD_{F} points which fail the lumen test are tagged as 0, while rest of the points are tagged as 1. All the AD_{F} points that tagged as 0 are deleted from the AD_{F} list.
3. The procedure is repeated for each AD_{F} point along the CA artery.

Note that even though, the lumen anatomic information, which acts as a reference, provides a good test for catching a series of wrongly computed ADF boundary, it might slip from sudden bumps which may be due to the changes in grayscale intensity due presence of unusual high intensity in lumen region or a calcium deposit in the near wall causing a shadow in far wall region. This sudden spike can then be easily detected ahead using the spike detection method.
Spike detection and removal. We implemented an intelligent strategy for spike detection and removal. Basically, we compute the first order derivative of the AD_{F} profile and check for values higher than 15 pixels. This value was chosen empirically by considering the image resolution. When working with images having approximate resolution of about 0.06 mm/pixel, an IMT value of 1 mm would be about 17 pixels. Therefore, a jump in the AD_{F} profile of the same order of magnitude of the IMT value is clearly a spike and error condition. If the spike is at the very beginning of the image (first 10 columns) or at the end (last 10 columns), then the spiky point is simply deleted. Otherwise, all spikes are considered and either substituted by a neighborhood moving average or removed.
Far adventitia AD_{F} automated tracing. Intensity profile for one column of the filtered image is then taken. The near and far walls clearly has intensity maxima saturated to the maximum value of 255. To automatically trace the profile of the far wall, we used a heuristic search applied to the intensity profile of each column. Starting from the bottom of the image (i.e. from the pixel with the higher row index), we searched for the first white region of at least 6 pixels of width. The deepest point of this region (i.e. the pixel with the higher row index) marked the position of the far adventitia (AD_{F}) layer on that column. The sequence of all the points of the columns constituted the overall AD_{F} automatically generated tracing.

Up-sampling to Fine Resolution. The AD_{F} profile is then up-sampled to the original scale and overlaid to the original image. At this stage, the carotid artery far wall is automatically located in the image frame and automated segmentation is made possible.

### LI/MA Border Segmentation (Stage II)

The goal for this stage in the segmentation process is the extraction of the LI and MA borders which lie in between the computed AD_{F} border and the lumen region. This region of the image in which the LI and MA borders can be found is called the guidance zone, which is empirically computed from the knowledge database. The mean shift algorithm is then run in the guidance zone, which classifies the image into three different classes based on pixel intensity. The borders between the three classes ideally represent the LI and MA borders but since the pixel intensity along the edge is not always uniform, this is often not the case. Therefore the final LI and MA profiles must undergo a refinement process which is based on dividing the borders into trends and a subsequent labeling process. The computed borders then undergo two final refinement checks by anatomic (lumen) reference and relative IMT measurement reference. This subsection is divided into four sections: Guidance Zone Mask Estimation; Regional Wall Segmentation using Mean Shift Classifier; LI/MA refinement process and Final refinement checks.

### Guidance Zone Mask Estimation

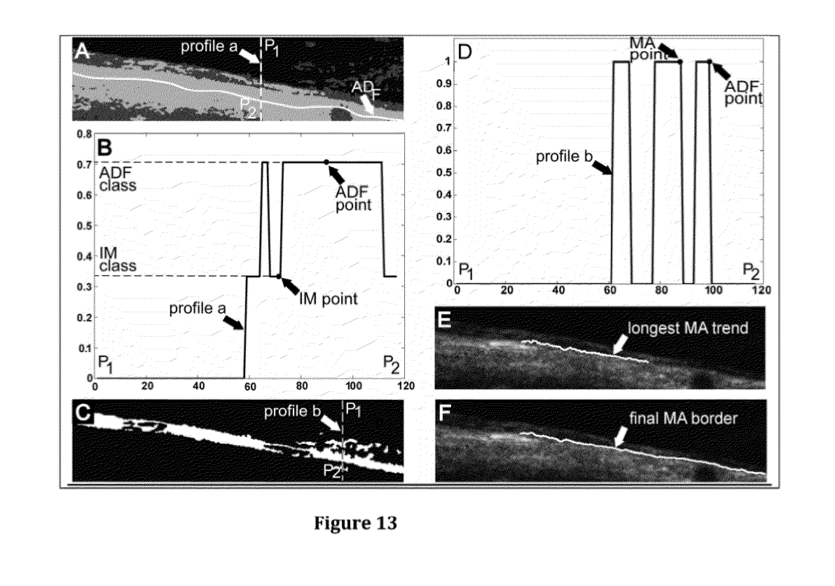

Stage II of the segmentation process is initialized by the automatic extraction of a guidance zone mask, which is found by starting from the computed AD_{F} profile and extending it upwards by Δ_{ROI}. This value to be equal to 50 pixels. This height is chosen after empirically computing the distances from the AD_{F} profile w.r.t. the ground truth LI/MA borders. As distance metric, Polyline Distance metric is used, which is fully described below. The mask consists of a binary image in which the white pixels correspond to those pixels that are included in the guidance zone. The original image is then cropped using the smallest rectangle possible that includes the entire guidance zone mask. Subsequently, the mean shift regional wall segmentation algorithm is run on the cropped grayscale image (FIG. 13) in order to obtain an initial classification of the wall regional image.

### Regional Wall Segmentation Using Mean Shift Classifier (Stage II)

The mean shift classifier used for feature space analysis as proposed by Comaniciu and Meer (Comaniciu and Meer, 1997), is a classification algorithm based on a simple and nonparametric technique for the estimation of the density gradient which was proposed originally by Fukunaga (Fukunaga, 1990) and subsequently generalized by Cheng (Cheng, 1995). This algorithm, whose complete description can be found in (Comaniciu and Meer, 1997), uses a search window of a certain radius r initialized in a chosen location. Inside this search window, the vector of difference between the local mean and the center of the window is calculated: the mean shift vector. The search window is then translated by the found amount, and finally these steps are repeated until convergence. A fundamental property of this mean shift vector is its proportionality to the gradient of the probability density at the considered point. The high density regions correspond to small mean shifts, while low density regions correspond to large mean shifts. In this way, the shifts are always in the direction of the mode, i.e., the probability density maximum. The mean shift algorithm can be used as a tool for any feature space analysis, and an outline of a general procedure is as follows:
(1) The image domain is mapped into the feature space.
(2) An adequate number of search windows are defined at random locations in the space.
(3) The high density region centers are found by applying the mean shift algorithm to each window.
(4) The extracted centers are validated with image domain constraints in order to provide the feature palette.
(5) All of the feature vectors are allocated to the feature palette using image domain information

Automatic image segmentation is thus obtained following the guidelines described above and is presented in full detail in the specific case of image segmentation in (Comaniciu and Meer, 1997). This technique is dependent on a minimal number of parameters. The first parameter is the most general parameter that characterizes a segmentation technique: segmentation resolution. The algorithm as implemented in (Comaniciu and Meer, 1997) distinguishes segmentation resolution into three important classes: under-segmentation corresponds to the lowest resolution in which the region boundaries are the dominant edges in the image; over-segmentation corresponds to intermediate resolution in which the image is broken into many small regions; quantization corresponds to the highest resolution which contains all of the important colors. The second and last parameter is the maximum number of colors (or gray tones in the case of a gray scale image) the image can be classified into. This parameter can also be thought of as the maximum number of classes that the MSC algorithm can distinguish. In our particular case, we chose to use the under-segmentation class since we are interested in finding the dominant edges in the image (i.e., the LI and MA profiles), and we chose an initial maximum number of gray tones equal to three. This value was specifically chosen since the goal is to classify three different regions of the considered guidance zone: the adventitia layer, the intima and media layers, and the lumen. As defined in (Comaniciu and Meer, 1997), the under-segmentation class is then translated into three parameters used in the mean shift algorithm:
(1) the radius of the search window, r, is taken to be equal to 4, defined as the square root of the global covariance matrix;
(2) the smallest number of elements that are required for a significant color, Nₘᵢₙ, is taken to be equal to 100;
(3) the smallest number of contiguous pixels required for a significant image region, N_{con}, is taken to be equal to 10.

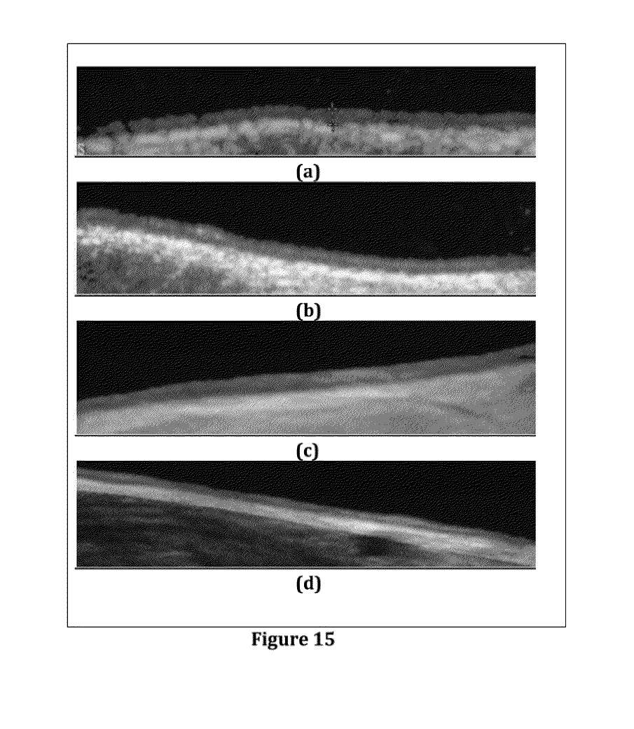

An example output image from the mean shift algorithm is displayed in FIG. 15 as colored overlay on the original grayscale Guidance Zone.

Ideally, the borders between the three found classes should represent the desired LI and MA profiles, but as FIG. 15 clearly shows, this is often not the case. In fact, ultrasound images are typically noisy images and can be affected by backscattering in the lumen. Another factor that prevents the use of this ideal solution (i.e., using the raw borders between the classes as the LI and MA profiles) is that the image was cropped so as to contain the entire guidance zone in a rectangle, and in many cases contains sections of the image that are found below the AD_{F} profile and/or farther above in the lumen. This problem is amplified in the case of inclined or curved arteries, since the rectangle containing the entire guidance zone assumes a more extended height and hence contains more tissue structures not found in the original guidance zone. These problems can lead to a jagged border between two classes, the presence of numerous small sections of one class enclosed inside a larger section of another class and/or a border that does not correctly run along the true LI and MA borders. It is therefore evident that a refinement process must be implemented before the final LI and MA borders can be defined.

### LI/MA Reconstruction (Stage II)

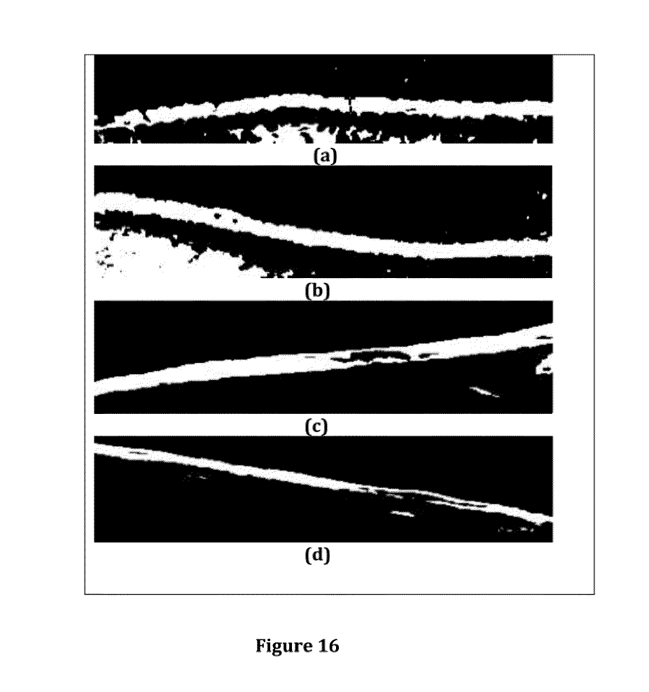

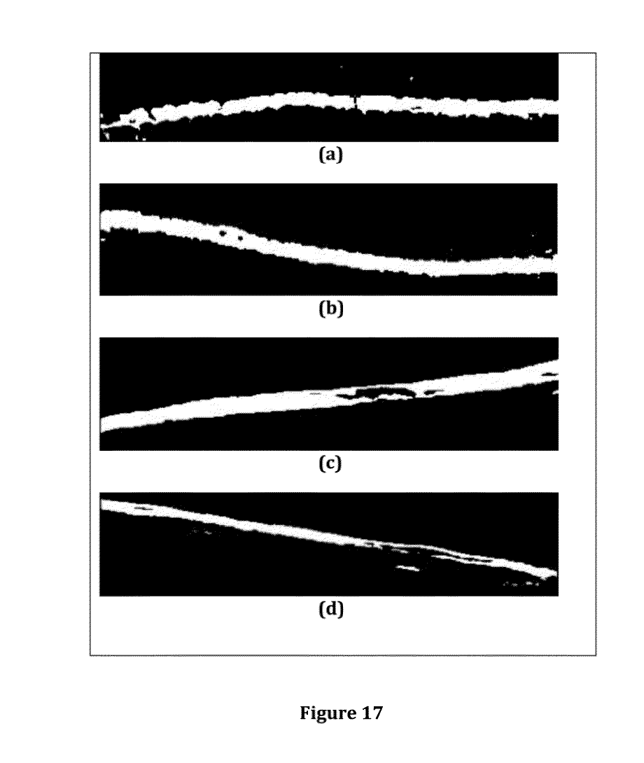

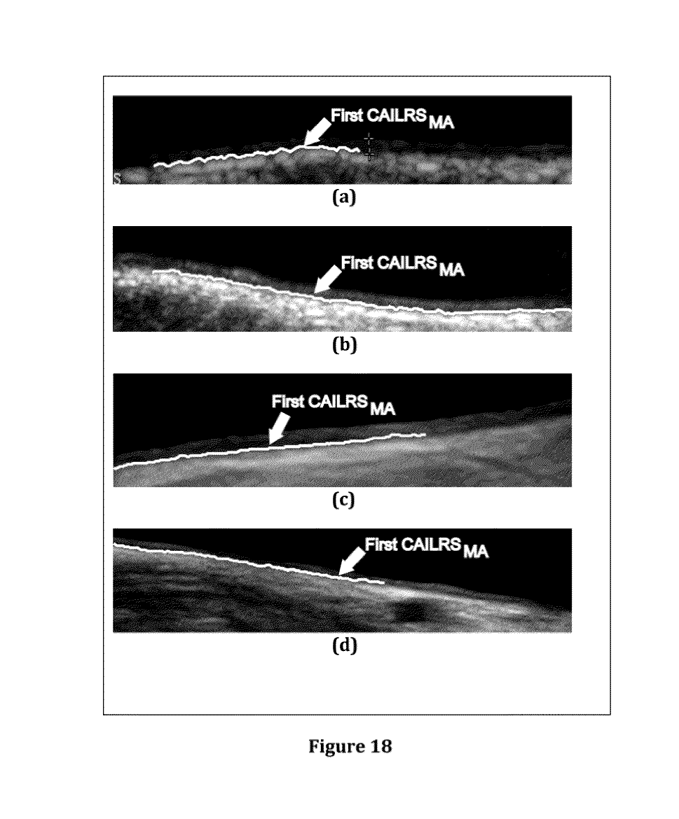

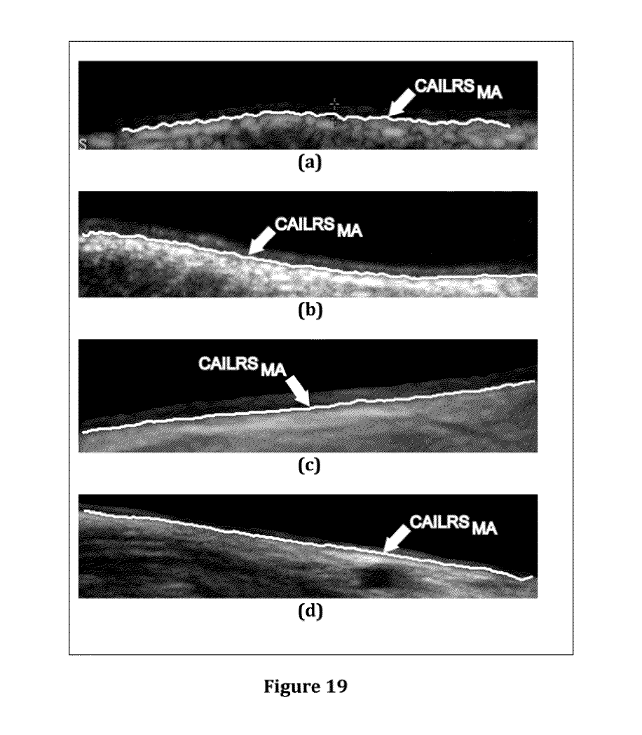

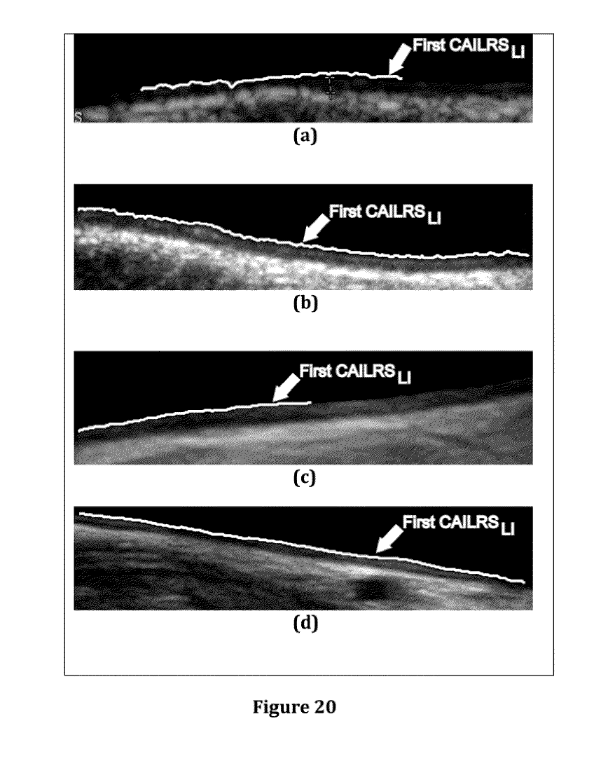

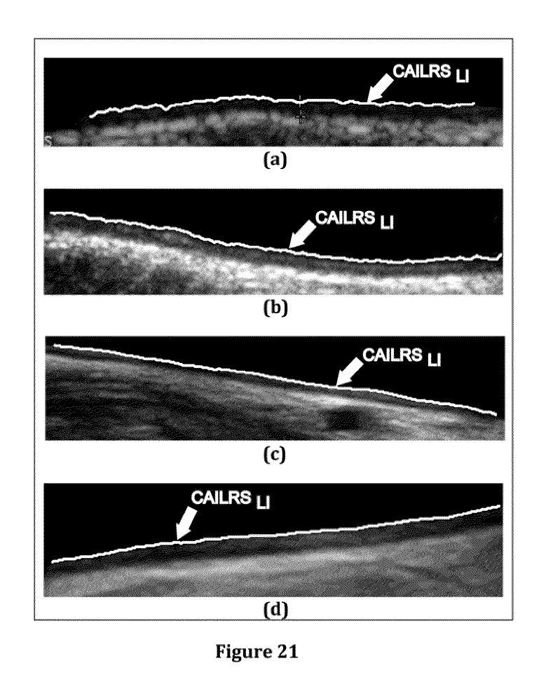

The first challenge in the refinement process is to automatically find the correct class that corresponds to the intima and media layers (IM_{class}). First of all, the class which contains the adventitia layer (AD_{F}_{_{class}}) is found by determining which classes correspond to each point of the AD_{F} profile, and then establishing the AD_{F}_{_{class}} as the most recurring class. Subsequently, the output image is examined column by column starting from the AD_{F} point and the class found at the first transition point is taken as the IM_{class} for that column. The final IM_{class} is then ascertained by finding the most recurring class. FIG. 16 reports the segmented IM_{class} as a binary (i.e., black and white) mask. A preliminary step of the refinement process is to remove all objects that are not included in the original guidance zone. This is useful so as to be sure that only sections of the image contained in the original region of interest are considered, and thus removes the problem of classifying unwanted tissue lying beneath the AD_{F} profile or too high above in the lumen (FIG. 17). After this step, exceedingly small objects (defined as those objects in the binary image which have an area (i.e., total number of connected pixels) less than 7) are eliminated. Our experimental data showed that is an optimal value to guarantee the removal of small objects that may remain after those not included in the guidance zone are eliminated, but to not discard relatively small objects of interest that are fundamental for establishing a correct MA or LI profile. A preliminary MA profile is then established by doing a column by column search for the first transition point from 0 to 1 found above the AD_{F} profile point for the considered column. This finds the first point belonging to the IM_{class} avoiding however including pixels that are found directly above the AD_{F} profile point. Once this profile is found, the following steps are implemented to determine the final MA profile:
(1) Divide the preliminary profile into different trends, where a single trend is defined as those points of the profile whose contiguous points respect the following equation:

|profie_{y}−profile_{y+1}|≦δ (1)
So, two contiguous points that have a vertical distance greater than δ are classified as belonging to two separate trends. This threshold was chosen to be equal to 3 pixels which correspond roughly to 0.2 mm, which is about one forth the value of a normal IMT. This value separates the profile into trends that are irregular but ensures the possibility of not distinguishing too many trends in the case of a curved vessel.
(2) Initialize the profile as the longest trend that is found (FIG. 18).
(3) Examine the remaining trends one by one and are classify them as belonging to the profile if two conditions are met. For the sake of clarity, let A and B be the two closest points of the separate trends.

|A_{y}−B_{y}|≦φ (2)

|Aₓ−Bₓ|≦φ (3)
The first check is to help avoid connecting a trend to the profile which is found too far above, while the second check is used to avoid connecting two trends that are too far apart from one another, which could cause errors in the final determination of the profile, especially in the case of inclined or curved vessels. Pilot studies we performed on our image dataset demonstrated that φ taken to be equal to 4 if the two points are contiguous or equal to 7 in any other case are suitable thresholds for linking two separate trends. 7 pixels approximately corresponds to 0.45 mm which is half of the normal IMT value and permits linking trends that are not right next to each other and perfectly aligned. The lower value in the case of trends that are next to each other was chosen to allow linking in the case of curved or inclined arteries but to preclude linking of a trend that is too far from the profile. Our studies showed instead that a suitable value for φ is equal to 20 pixels.
(4) B-spline fit the different trends to provide the final profile (FIG. 19).
The LI border that is subsequently established is strongly dependent on the computed MA border, since the preliminary LI profile is found by doing a column by column search starting from the MA point of the considered column. Therefore, only the columns where a MA point was found are considered. The LI point for each column is determined as the first transition point from 1 to 0. A check is done on the distance between the MA point and the found LI point to be sure to skip over potential small holes found in the binary image which are too near to the MA border.
   The final LI profile is then ascertained by following the same 4 steps described in the previous subsection for the determination of the MA profile (FIGS. 20, 21).

### LI/MA Refinement and Checks (Stage II)

Once the two borders of interest are computed, two final checks are carried out. The first check, by anatomic (lumen) reference, is to avoid the potential error case in which the computed LI profile falls inside the lumen area. This error can be due to the fact that the intima and media layers acquire a dark aspect in the ultrasound image and therefore the MSC classifier correctly identifies the two classes whose border gives the MA profile but associates the pixels belonging to the artery wall as part of the lumen, and therefore does not correctly identify the LI border. So to prevent this, the same lumen validation process as described herein is used. The computed LI profile is therefore defined as falling inside the lumen area if more than 25% of the LI points are classified as belonging to the lumen area of the vessel as determined by the support routine. If this should be the case, the MSC algorithm is rerun on the image, but this time using a maximum number of classes equal to 4 (compared to the original maximum number, 3) and reducing the guidance zone. Since the dark aspect of the intima and media layers may also polarize the performance of the lumen validation process, the reduced guidance zone is initialized as solely the portion of the image that is included in the original guidance zone and that is not classified as the lumen area by the support routine. Then a column by column search is done to verify that the upper limit of the new guidance zone of the considered column is found at least 15 pixels above the MA point for the same column. If this is not verified, the upper limit for such column is empirically, imposed to be 15 pixels above the MA point, ensuring therefore that it encloses the vessel wall. This value was considered optimal after pilot studies on our database and in fact, 15 pixels Corresponds to approximately 1 mm, a value which is slightly higher than the normal IMT value. This ensures that the entire vessel wall is included in the new guidance zone without stretching too far into the lumen area. The second check, by relative IMT measurement reference, is to avoid the potential error case in which the MSC classifier mistakenly associates a large section of the intima and media layer as belonging to the adventitia layer. This causes a computed MA profile which incorrectly lies in between the true MA and LI borders. To remedy this, the following distances are calculated:

D₁=PD(MA,AD_{F}) (4)

D₂=PD(LI,AD_{F}) (5)

The ratio

$$\frac{{D}_{1}}{{D}_{2}}$$

is subsequently calculated, which gives a relative measure of the IMT value, therefore avoiding imposing an absolute cut-off value which could prove troublesome in the case of different image resolutions. So, if the computed MA border is found in between the two border walls, the calculated ratio increases with respect to a correct case since D1 is higher. Pilot studies performed on our image database showed that, even in the case of irregular AD_{F} profiles, images in which the LI and MA borders were correctly identified produced a

$$\frac{{D}_{1}}{{D}_{2}}$$

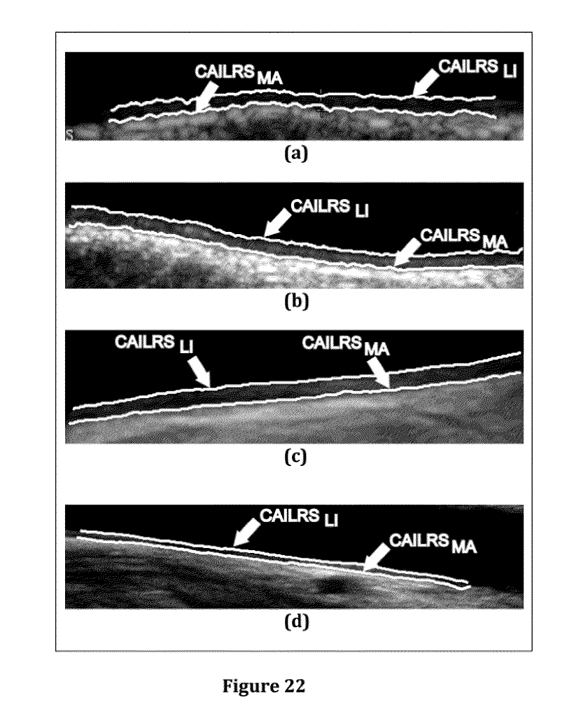

ratio lower than 0.6. This value was taken as a cut-off limit for this check. If an image does not pass this test, the MSC algorithm is rerun on the image, this time using a maximum number of gray tones, equal to 4 while still keeping the original guidance zone size.
As described above, both of these cases for which a final refinement check is required are due to an incorrect initial classification, and our method for resolving the problem consists in raising the number of classes which the mean shift classifier can distinguish. This extra class allows the classifier to distinguish the two separate classes that originally were merged together in one. The final LI and MA borders are represented by FIG. 22.

### DETAILED DESCRIPTION OF AN EXAMPLE EMBODIMENT

In the following description, for purposes of explanation, numerous specific details are set forth in order to provide a thorough understanding of the various embodiments. It will be evident, however, to one of ordinary skill in the art that the various embodiments may be practiced without these specific details.

This patent application discloses a computer-based system and method for intima-media thickness (IMT) measurements in presence of calcium or absence of calcium in near (proximal) end of the arterial value. The embodiment is being designed for carotid, femoral, brachial and aortic arteries. IMT measurement is a very important risk marker of the Atherosclerosis disease. Typically, there are two ways to measure the arterial IMT's: (a) invasive methods and (b) non-invasive methods. In invasive methods, traditionally, intravascular ultrasound (IVUS) is used for measuring vessel wall thickness and plaque deposits where special catheters are inserted in the arteries to image them. Conventional ultrasound is used for measuring IMT non-invasively, such as from carotid, brachial, femoral and aortic arteries. The main advantages of non-invasive methods are: (i) low cost; (ii) convenience and comfort of the patient being examined; (iii) lack of need for any intravenous (IV) insertions or other body invasive methods (usually), and (iv) lack of any X-ray radiation; Ultrasound can be used repeatedly, over years, without compromising the patient's short or long term health status. Though conventional methods are generally suitable, conventional methods have certain problems related to accuracy and reliability.

The IMTs are normally 1 mm in thickness, which nearly corresponds to 15 pixels on the screen or display. IMT estimation having a value close to 1 mm is a very challenging task in ultrasound images due to large number of variabilities such as: poor contrast, orientation of the vessels, varying thickness, sudden fading of the contrast due to change in tissue density, presence of various plaque components in the intima wall such as lipids, calcium, hemorrhage, etc. Under normal resolutions, a 1 mm thick media thickness is difficult to estimate using stand-alone image processing techniques. Over and above, the image processing algorithms face an even tighter challenge due to the presence of speckle distribution. The speckle distribution is different in nature from these interfaces. This is because of the structural information change between intima, media and adventitia layers of the vessel wall. As a result, the sound reflection from different cellular structures is different. The variability in tissue structure—all that happens in 1 mm of the vessel wall—brings fuzziness in the intensity distribution of the vessel wall. Under histology, media and adventitia walls are clearly visible and one can observe even their thicknesses. This 1 mm zone is hard to discern in a normal resolution image of 256×256 pixels in a region of interest (ROI) or in a higher resolution image of 512×512 pixels in a region of interest (ROI). One needs a high resolution image to process and identify the intensity gradient change in ultrasound images from lumen to intima and media to adventitia layers. The ultrasound image resolution may not be strong enough like MRI or computerized axial tomography (CAT or CT) images, which can be meaningful for soft tissue structural information display.

There are two ways to process and identify the intensity gradient change in ultrasound images from lumen to intima (LI) and media to adventitia (MA) layers: (a) have a vascular surgeon draw the LI/MA borders and compute the NT image interactively, OR (b) have a computer determine the LI and MA borders along with IMT's. Case (a) is very subjective and introduces variability in the IMT estimation. IMT screenings are really part of the regular check-up for patients and millions of scans are done each day around the world. The manual handling of such a repetitive work flow of IMT screenings is tedious, error-prone and subject to lot of variability. Case (b) is difficult to implement, because it is difficult to identify the LI and MA borders with heavy speckle distribution and the inability of ultrasound physics to generate a clear image where the semi-automated or automated image processing methods are used for IMT estimation. Besides that, the calcium deposit in the near walls causes the shadow.

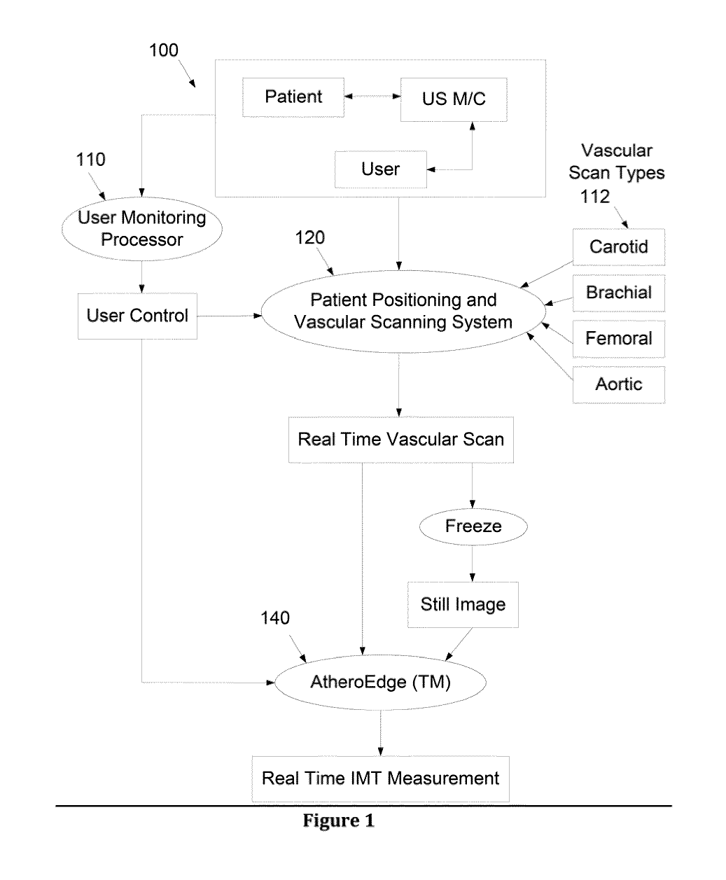

FIG. 1 shows how the main system uses AtheroEdge™ processor. The system shows how the ultrasound vascular image is acquired using the longitudinal B-mode process (block 120). The input to the system also shows that this process can take any of the four arteries: carotid, brachial, femoral and arotic (block 112). The system has ability to, freeze the image as a still image, on which the IMT will be computed. User continuously monitors the process at all stages during the operation (block 110). User has control on the AtheroEdge™ software system, ultrasound machine, ultrasound probe, patient and the graphical user interface (block 110). The still image can be saved on the hard drive or CD drive. The still image-can then also be transferred to an independent computer and AtheroEdge™ can be run on that system as well. At the same time AtheroEdge™ can run real time while the patient is in the vascular screening room.

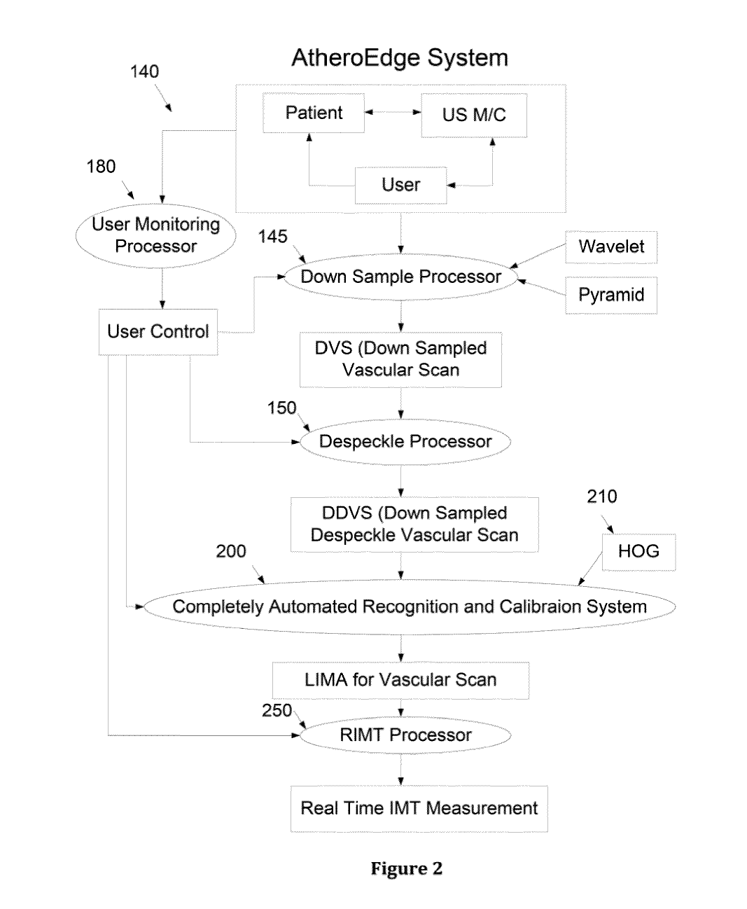

FIG. 2 shows the AtheroEdge™ system where the main components of the system are: (a) Multi-resolution Image Processor (block 145); (b) De-speckle Processor (block 150); (c) Recognition, Validation Processor and Calibration Processor (block 120 using higher order Gaussian derivative operators (HOG) block 210) and (d) RIMT Processor (block 250).

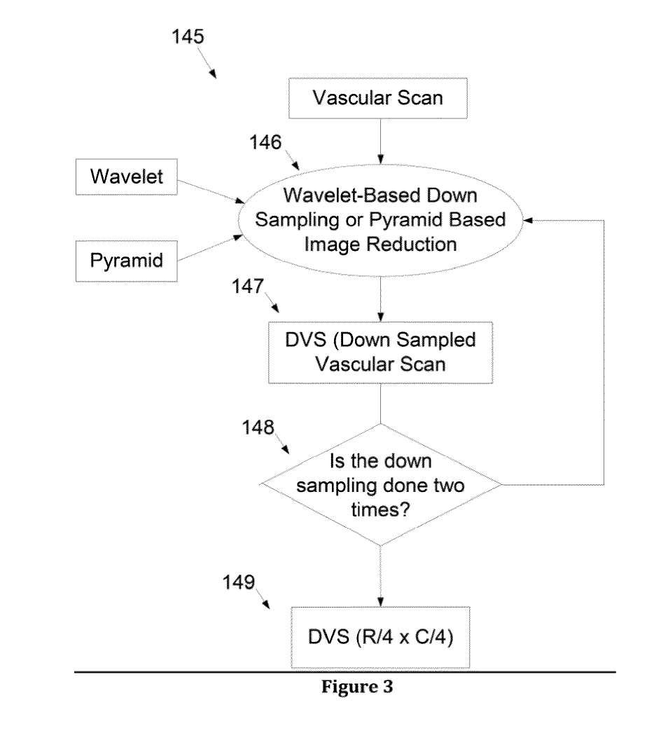

Multi-resolution image processing yields the DSVS (down sampled vascular scan) image. FIG. 3 shows the down sampling or fine to coarse resolution system (block 145). One of the four systems can be used for fine to coarse sampling. The role of the multi-resolution process is to convert the image from fine resolution to coarse resolution. Those skilled in the art of down sampling any use off the shelf down sampling methods. One of the very good down samplers are Lanczos interpolation. This is based on the sin c function which can be given mathematically as

$$sin c ( x ) = \frac{sin ( π x )}{π x} .$$

Since the sin c function never goes to zero, practical filter can be implemented by taking the sin c function and multiplying it by a “window”, such as Hamming and Hann, giving an overall filter with finite size. We can define the Lanczos window as a sin c function scaled to be wider, and truncated to zero outside of the main lobe. Therefore, Lanczos filter is a sin c function multiplied by a Lanczos window. Three lobed Lanczos filter can be defined as

$$Lanczos 3 ( x ) = \{ \begin{matrix}\frac{sin ( π x ) sin ( π \frac{x}{3} )}{π x \cdot π \frac{x}{3}} , & if \vert x \vert \leq 3 \\ 0 , & if \vert x \vert > 3\end{matrix}$$

Although Lanczos interpolation is slower than other approaches, it can obtain the best interpolation results because Lanczos method attempts to reconstruct the image by using a series of overlapping sin c waves to produce what's called a “best fit” curve. Those skilled in the art of down sample can also use Wavelet transform filters as they are very useful for multi-resolution analysis. The orthogonal wavelet transform of a signal f can be formulated by

$$f ( t ) = {\sum }_{k ∈ z} {c}_{j} ( k ) {ϕ}_{j , k} ( t ) + {\sum }_{j = 1}^{J} {\sum }_{k ∈ z} {d}_{j} ( k ) {ϕ}_{j , k} ( t )$$

where the cⱼ(k) is the expansion coefficients and the dⱼ(k) is the wavelet coefficients. The basis function φ_{j,k}(t) can be presented as

φ_{j,k}(t)=2^{-j/2}φ(2^{-j}t−k),
where k, j are translation and dilation of a wavelet function φ(t). Therefore, wavelet transforms can provide a smooth approximation of ƒ(t) at scale J and a wavelet decomposition at per scales. For 2-D images, orthogonal wavelet transforms will decompose the original image into 4 different sub-band (LL, LH, HL and HH) (block 149).

Bicubic interpolation can also be used as it will estimates the value at a given point in the destination image by an average of 16 pixels surrounding the closest corresponding pixel in the source image. Given a point (x,y) in the destination image and the point (l,k) (the definitions of l and k are same as the bilinear method) in the source image, the formulae of bicubic interpolation is

$$f ( x , y ) = {\sum }_{m = l - 1}^{l + 2} {\sum }_{n = k - 1}^{k + 2} g ( m \cdot n ) \cdot r ( m - l - dx ) \cdot ( dy - n + k ) ,$$

where the calculation of dx and dy are same as the bilinear method. The cubic weighting function r(x) is defined as

$$r ( x ) = \frac{1}{6} [ {p ( x + 2 )}^{3} - 4 {p ( x + 1 )}^{3} + 6 {p ( x )}^{3} - 4 {p ( x - 1 )}^{3} ] ,$$

where p(x) is

$$p ( x ) = \{ \begin{matrix}x & x > 0 \\ 0 & x \leq 0\end{matrix}$$

Bicubic approach can achieve a better performance than the bilinear method because more neighboring points are included to calculate the interpolation value.

Bilinear interpolator can also be used as it is very simple to implement. Mathematically, it is given as: if g represents a source image and f represents a destination image, given a point (x,y) in f, the bilinear method can be presented as:

ƒ(x,y)=(1−dx)·(1−dy)·g(l,k)+dx·(1−dy)·g(l+1,k)+(1−dx)·dy·g(l,k+1)+dx·dy·g(l+1,k+1),

where l=└x┘ and k=└y┘, and the dx dy are defined as dx=x−l and dy=y−k respectively. Bilinear interpolation is simple. However it can cause a small decrease in resolution and blurring because of the averaging nature.

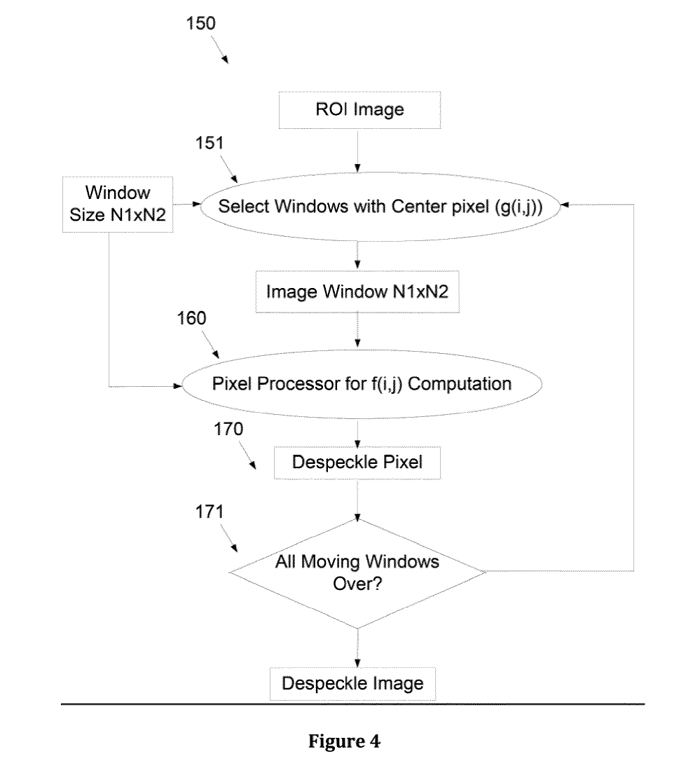

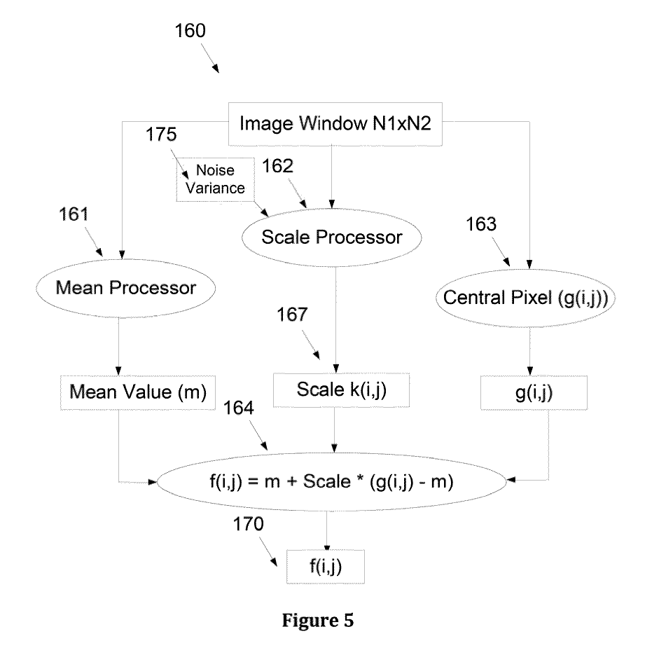

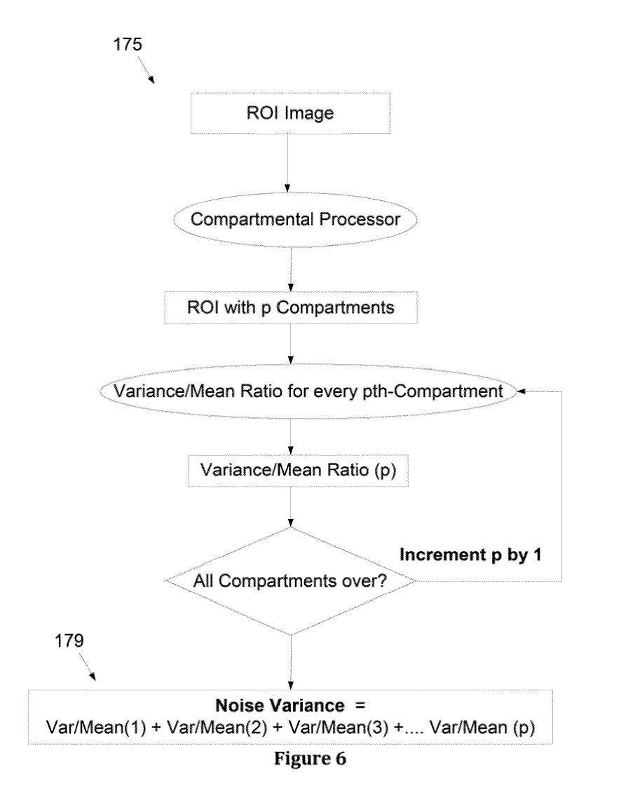

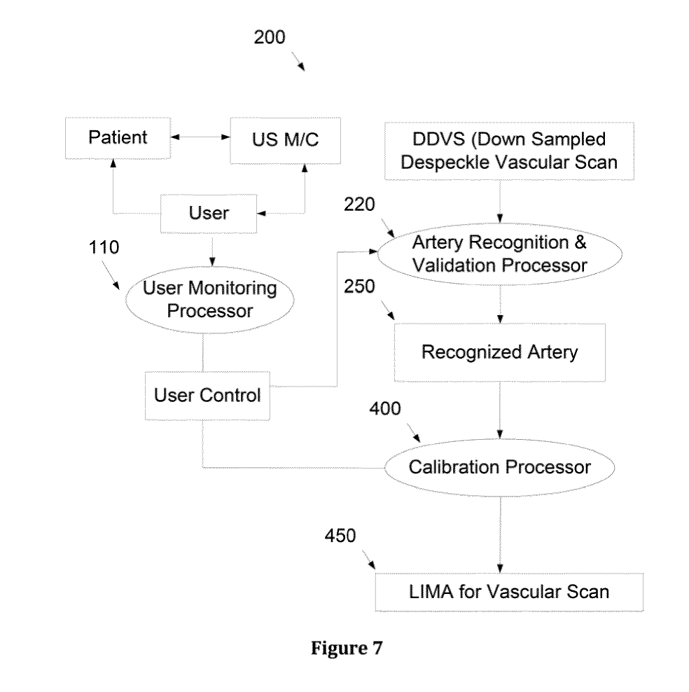

FIGS. 4, 5 and 6 deal with the de-speckle filtering; whose output is DDVS (Down sampled Despeckle Vascular Scan). Speckle noise was attenuated by using a first-order statistics filter (block 160), which gave the best performance in the specific case of carotid imaging. This filter is defined by the following equation (block 164):

J_{x,y}=Ī+k_{x,y}(I_{x,y}−Ī) (1)

where, I_{x,y} is the intensity of the noisy pixel, Ī is the mean intensity of a N×M pixel neighborhood and k_{x,y} is a local statistic measure. The noise-free pixel is indicated by J_{x,y}. k_{x,y} is mathematically defined

$${k}_{x , y} = \frac{{σ}_{I}^{2}}{{\overline{I}}^{2} {σ}_{I}^{2} + {σ}_{n}^{2}} ,$$

where σₗ² represents the variance of the pixels in the neighborhood, and σₙ² the variance of the noise in the cropped image (block 175 and 179). An optimal neighborhood size can be 7×7. Note that the despeckle filter is useful in removing the spurious peaks if any during the adventitia identification in subsequent steps.

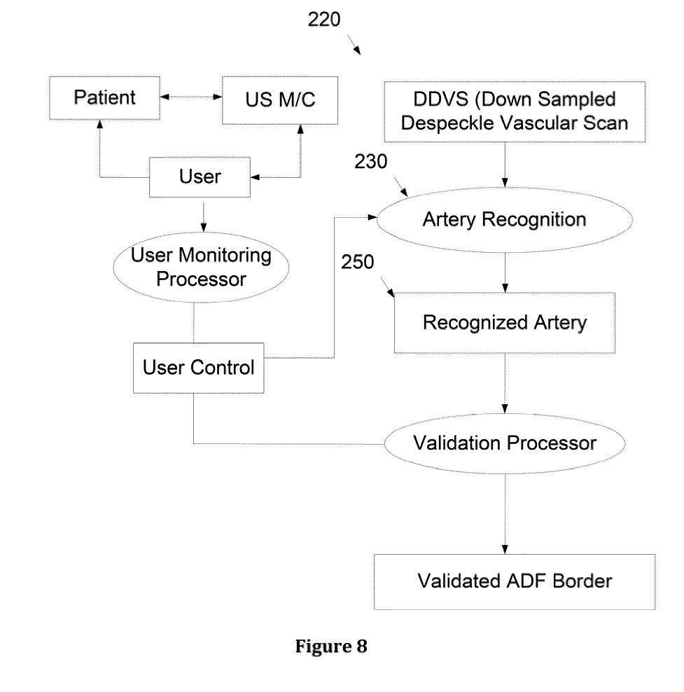

FIG. 8 shows the last and the final stage is the recognition, validation and calibration system shown in the process called “Completely Automated Recognition and Calibration Processor”. While the two stages are cascaded and shown to be different blocks, but it is transparent to the user. This means the software is cascading information from one block to another block without user interaction. The user still has full control and user monitoring processor is fully active and the user can interrupt the system any time. It shows two stages: stage I is the Artery recognition process (block 220) and calibration processor (block 400).

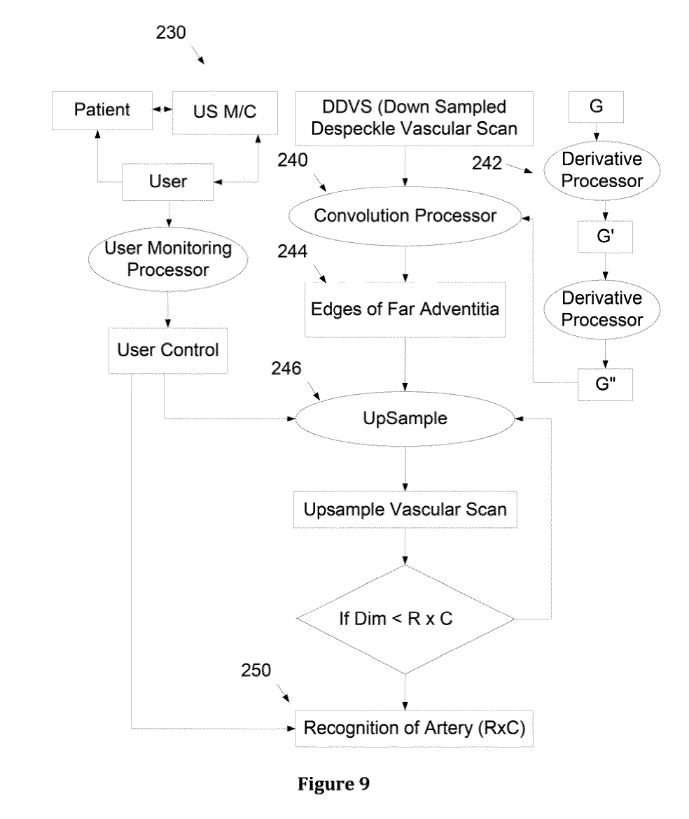

FIG. 9 shows the Artery Recognition Processor (block 220), which is the novelty of the patent as it does the automated recognition. It has two stages: (a) convolution and heuristic processor and (b) up-sample processor.

The convolution processor (block 240) is used for convolution of the first order derivative G (block 242) with the despeckled image. Those skilled in the art can use higher order derivatives as well. The scale parameter of the Gaussian derivative kernel was taken equal to 8 pixels, i.e. to the expected dimension of the IMT value. In fact, an average IMT value of say 1 mm corresponds to about 16 pixels, in the original image scale and, consequently, to 8 pixels in the coarse or down sampled image. The convolution processor outcome will lead to the clear information for the near and far walls. This information will have two parallel bands corresponding to the far and near vessel walls. These bands will follow the curvature of the vessel walls. If the vessel wall is oriented downwards or upwards or has a bending nature, the bands will follow on both sides of the lumen. These bands have information which corresponds to the maximum intensity saturated to the maximum values of 2 power 8, the highest value. For an 8 bit image, this value will be 255.

The convolution process then allows the heuristics to estimate the Adventitia borders of the far wall or near wall (block 244). To automatically trace the profile of the far wall, this application uses heuristic search applied to the intensity profile of each column. Starting from the bottom of the image (i.e. from the pixel with the higher row index. The image convention uses (0,0) as top left hand corner of the image), we search for the first white region constituting of at least 6 pixels of width. The deepest point of this region (i.e. the pixel with the higher row index) marked the position of the far adventitia (ADF) layer on that column. The sequence the points resulting from the heuristic search for all the image columns constituted the overall automated far adventitia tracing ADF. The ADF is up sampled back to the original scale (block 246).

FIG. 9 shows the Artery Validation Processor. Pilot studies showed that the traced ADF profile could be characterized by spikes and false points identification. This could be due to several reasons such as variations in intensities, gaps in the media walls, presence of jugular vein, shadow effects or combination of these. We have therefore introduced a validation protocol, which provides a check on the ADF profile ensuring that the location of CA is at correct place and the far adventitia segmentation edge is not very bumpy. This validation step refines the ADF profile and is done in two steps: (a) refinement using anatomic lumen and (b) spike removal.

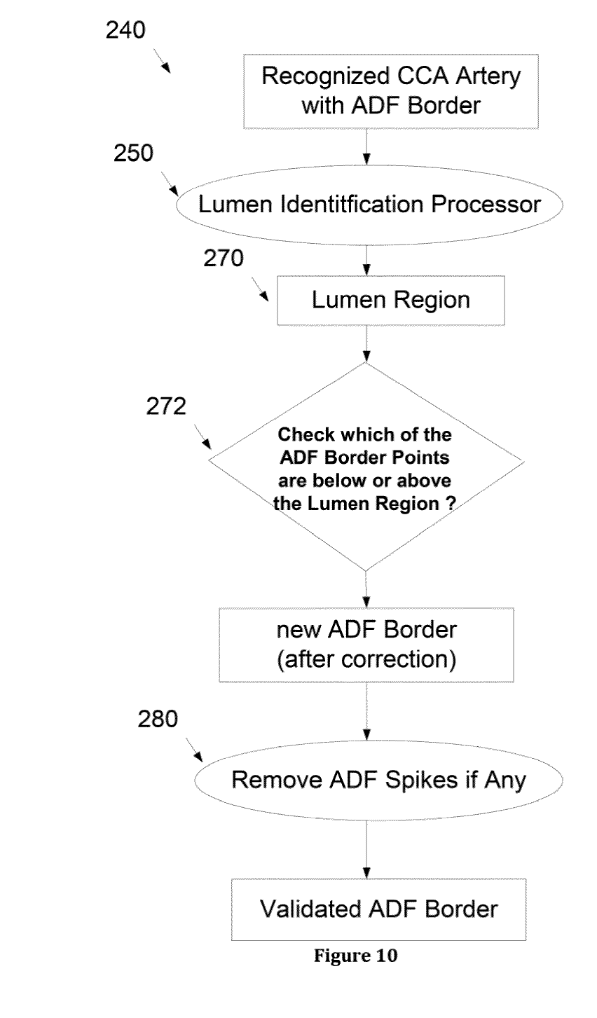

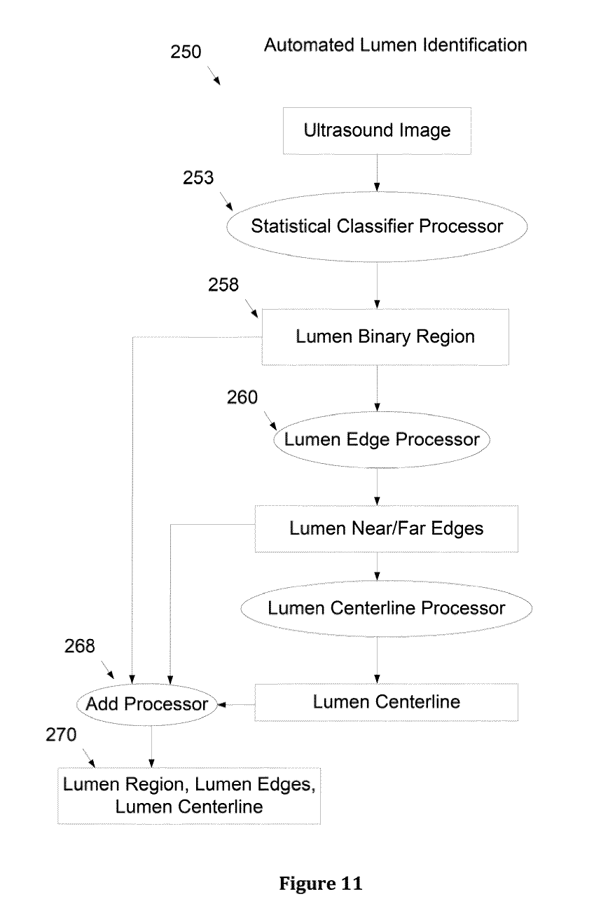

FIG. 10 & FIG. 11 shows the Check system (block 240), which has Lumen Identification Processor (block 250). This check (block 272) has been introduced to avoid error conditions of ADF profile protruding into the lumen vessel or beyond. Thus, the refinement step first requires the identification of the lumen region automatically. We have modeled the lumen segmentation region as a classification process (block 253) with two classes. Carotid characteristics can be thought of as a mixture model with varying, intensity distributions. This is because (a) the pixels belonging to the vessel lumen are characterized by low mean intensity and low standard deviation; (b) pixels belonging to the adventitia layer of the carotid wall is characterized by high mean intensity and low standard deviation; and (c) all remaining pixels should have high mean intensity and high standard deviation. As a result of this distribution, we derived a bi-dimensional histogram (2DH) of the carotid image (block 256). For each pixel, we considered a 10×10 neighborhood of which we calculated the mean value and the standard deviation. The mean values and the standard deviations were normalized between 0 and 1 and were grouped into 50 classes each having an interval of 0.02 (block 257). The number of classes equal to 50 and the corresponding class width of 0.02 had bee optimized in a previous study. The 2DH was then a joint representation-of the mean value and standard deviation of each pixel neighborhood.

In previous studies, we showed that pixels belonging to the lumen of the artery are usually classified into the first few classes of this 2DH: expert sonographer manually traced the boundaries of the CCA lumen and observed the distribution of the lumen pixels on the 2DH. Overall results revealed that pixels of the lumen have a mean values classified in the first 4 classes and a standard deviation in the first 7 classes. We therefore consider a pixel as possibly belonging to the artery lumen if its neighborhood intensity is lower than 0.08 and if its neighborhood standard deviation is lower than 0.14. This shows how the local statistic is effective in detecting image pixels that can be considered as belonging to the CCA lumen. This segmented lumen region act as a check point for the ADF profile estimated before. We therefore utilize the lumen region as follows:

The ADF points along the CA are considered one by one. For each ADF point:
(a) Region of Interest Estimation (ROI_{L}): We consider the sequence of the 30 pixels above it (i.e., the 30 pixels located above the AD_{F} point, towards the top of the image, and, therefore, with lower row indexes).
(b) Failure of AD_{F} profile point: We test if the ROI_{L} drawn around the AD_{F} profile points cross the lumen region and have penetrated into the lumen region by at least 15 pixels or more (let's indicate this threshold value by T_{L}). If this does not happens, then the AD_{F} profile point is considered to have failed the lumen test Pilot experiments we conducted revealed that suitable values for T_{L} are comprised between 12 and 20 pixels.
(c) Tagging of Profile Points: These failed AD_{F} profile points must not belong to the AD_{F} boundary. These AD_{F} points which fail the lumen test are tagged as 0, while rest of the points are tagged as 1. AD_{F} All the AD_{F} points that tagged as 0 are deleted from the AD_{F} list.
(d) The procedure is repeated for each AD_{F} point along the CA artery.

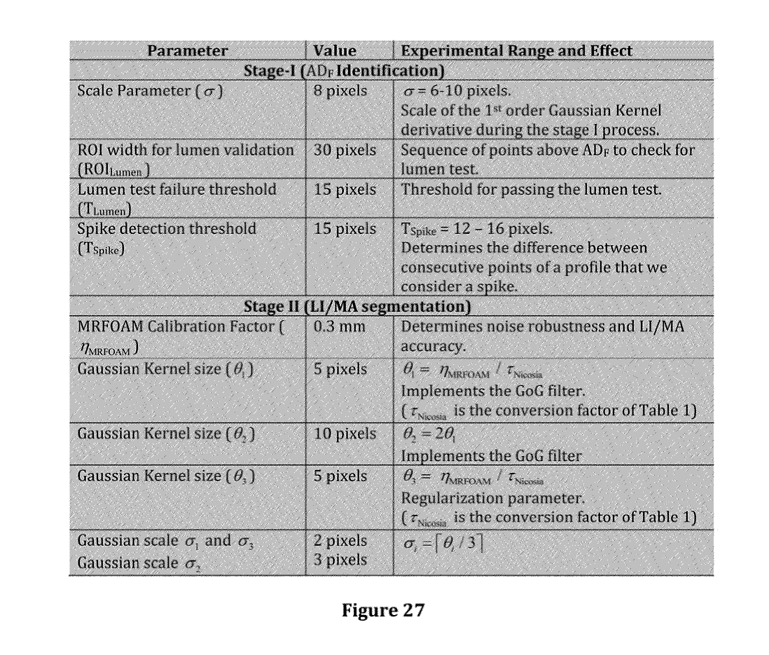

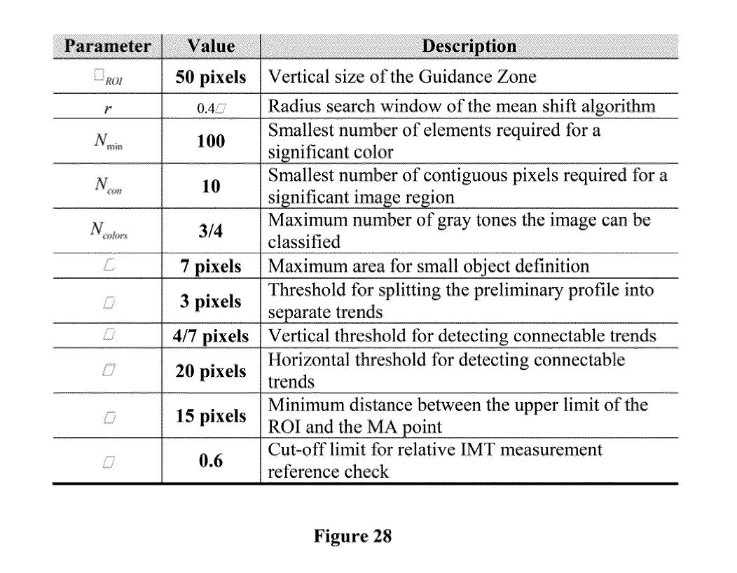

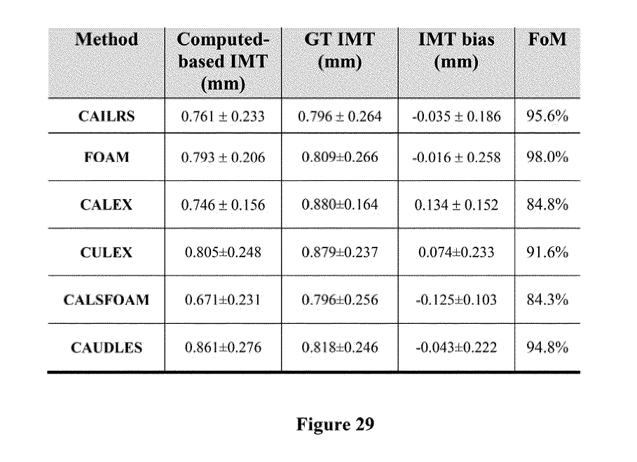

Table in FIG. 27 summarizes all the thresholds and parameters we used in AtheroEdge™. Table 2 in FIG. 28 summarizes the parameters used stage I (Mean Shift Classifier, LI/MA reconstruction and LI/MA refinement).

### Spike Detection and Removal.

We implemented an intelligent strategy for spike detection and removal. Basically, we compute the first order derivative of the ADF profile and check for values higher than TS=15 pixels. This value was chosen empirically by considering the image resolution. When working with images having approximate resolution of about 0.06 mm/pixel, an IMT value of 1 mm would be about 12-16 pixels. Therefore, a jump in the ADF profile of the same order of magnitude of the IMT value is clearly a spike and error condition. If the spike is at the very beginning of the image (first 10 columns) or at the end (last 10 columns), then the spiky point is simply deleted. Otherwise, all spikes are considered and either substituted by a neighborhood moving average or removed.

The last stage of the Artery Recognition Processor is the up-sampling processor which allows the adventitia tracing ADF to be up-sampled back to the original scale of cropped image. The ADF profile was then up-sampled to the original scale and superimposed over the original cropped image for both visualization and determination of the region of interest for segmentation (or calibration) phase. At this stage, the CA far wall is automatically located in the image frame and automated segmentation is made possible.

This Artery Recognition Processor (stage-I) is the most innovative aspect of our methodology. It consists of a superior architecture based on fine to coarse sampling for vessel wall scale reduction, speckle noise removal, and higher-order Gaussian convolution, and automated validation embedded recognition of Adventitia. The ability of segmentation or calibration phase (stage-II) to be guided by the automated CA wall recognition is in itself a novel contribution. The first-order Gaussian kernel convolution allowed for an optimal detection of the CA walls. This kernel has unitary energy. When such kernel is located in proximity of a neat gray level change, it enhances the transition. Consequently, the most echoic image interfaces are enhanced to white in the filtered image. For this reason, the Artery Recognition Processor allows for detecting the adventitia layer. This Artery Recognition Processor several advantages to it:
(1) Robustness and Accurate Wall Capture: it is very, robust because the higher order derivative kernels are very good in capturing the vessel walls (see, A Review on MR Vascular Image Processing Algorithms: Acquisition and Pre-filtering: Part I, Suri et al., IEEE TRANSACTIONS ON INFORMATION TECHNOLOGY IN BIOMEDICINE, VOL. 6, NO. 4, pp. 324-337, DECEMBER 2002; and A Review on MR Vascular Image Processing: Skeleton Versus Nonskeleton Approaches: Part II, Suri et al., IEEE TRANSACTIONS ON INFORMATION TECHNOLOGY IN BIOMEDICINE, VOL. 6, NO. 4, DECEMBER 2002).
(2) Faster than the conventional processing: Since the recognition is strategized at coarse level down sampled twice from its original size of the image, it is therefore processing ¼^{th} the number of pixels for automated recognition of the media layer. This improves the speed of the system.
(3) Independent of Orientation of the vascular scan: Another major advantage to the system is that these Gaussian kernels are independent of the orientation of the blood vessels in the image. Since the ultrasound vascular scans do not always have the vessel orientation horizontal with respect bottom edge of the image, manual methods can pose a further challenge towards the region of interest estimation.
(4) Guiding Method for the Calibration System: Since the recognition is followed by the calibration process, the calibration system becomes very robust since the calibration processing is done in the region of interest determined by the automated recognition system. Thus the calibration system adds the value determined by the automated recognition system for vascular ultrasound such as IMT measurement for carotid, femoral, aortic and brachial. Such a combination where the calibration system is guided by the automated recognition system helps in mass processing of huge database processing.
(5) Running the Mass IMT system for Clinical Analysis: Since the recognition is automated followed by the calibration system, the largest value such a system would deliver will be in its real time use for analysis of IMT measurement on a large databases. Running clinical databases on still images would be even more beneficial because such a system would be completely automated in terms of recognition and IMT measurement.
(6) Applications: Since the ultrasound probes use almost the same frequency of operation for scanning the vascular arteries such as carotid, femoral, brachial and aortic, it is thus possible to use such a system for these blood vessels.

### Calibration Processor (Stage II)

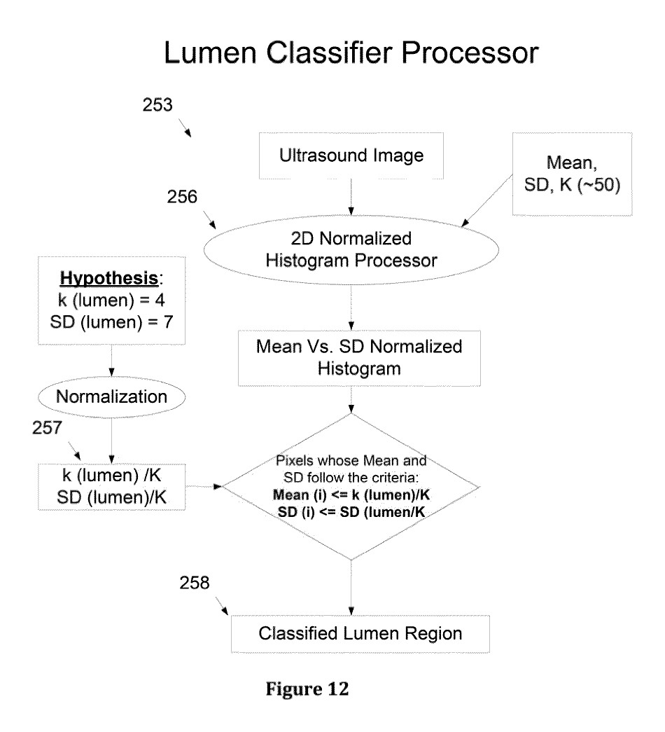

FIG. 12 shows the Calibration Processor (block 400). The output of the multi-resolution strategy provides the automated recognition of the distal wall in the carotid B-mode ultrasound images. Stage-II is focused narrowly in the region of interest, where the objective is to estimate the LI/MA borders accurately. It consists of (a) Mean Shift Classification Processor (block 420); (b) LI/MA reconstruction and Refinement Processor (block 440) and (c) Smoothing Processor (block 460). The goal of stage II i.e., segmentation process is the extraction of the LI and MA borders which lie in between the computed AD_{F} border and the lumen region. This region of the image in which the LI and MA borders can be found is called the guidance zone, which is empirically computed from the knowledge database. The mean shift algorithm is then run in the guidance zone, which classifies the image into three different classes based on pixel intensity. The borders between the three classes ideally represent the LI and MA borders but since the pixel intensity along the edge is not always uniform, this is often not the case. Therefore the final LI and MA profiles must undergo a refinement process which is based on dividing the borders into trends and a subsequent labeling process. The computed borders then undergo two final refinement checks by anatomic (lumen) reference and relative IMT measurement reference. This subsection is divided into four ulterior sections:

### Guidance Zone Mask Estimation

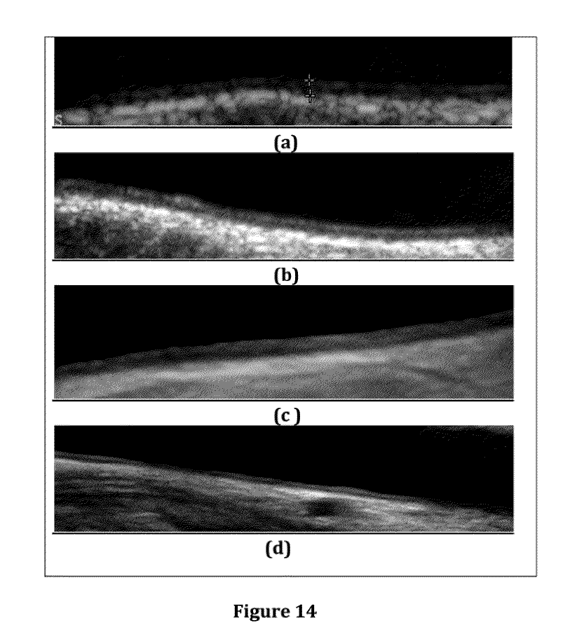

Stage II of the segmentation process is initialized by the automatic extraction of a guidance zone mask, which is found by starting from the computed AD_{F} profile and extending it upwards by Δ_{ROI}. We set this value to be equal to 50 pixels. We chose this height after empirically computing the distances from the AD_{F} profile w.r.t. the ground truth LI/MA borders. As distance metric we used the Polyline Distane, which is fully described below. The mask consists of a binary image in which the white pixels correspond to those pixels that are included in the guidance zone. The original image is then cropped using the smallest rectangle possible that includes the entire guidance zone mask. Subsequently, the mean shift regional wall segmentation algorithm is run on the cropped grayscale image (FIG. 14) in order to obtain an initial classification of the wall regional image.

Regional Wall Segmentation using Mean Shift Classifier

The mean shift classifier used for feature space analysis as proposed by Comaniciu and Meer (Comaniciu and Meer, 1997), is a classification algorithm based on a simple and nonparametric technique for the estimation of the density gradient which was proposed originally by Fukunaga (Fukunaga, 1990) and subsequently generalized by Cheng (Cheng, 1995). This algorithm, whose complete description can be found in (Comaniciu and Meer; 1997), uses a search window of a certain radius r initialized in a chosen location. Inside this search window, the vector of difference between the local mean and the center of the window is calculated: the mean shift vector. The search window is then translated by the found amount, and finally these steps are repeated until convergence. A fundamental property of this mean shift vector is its proportionality to the gradient of the probability density at the considered point. Thanks to this property, high density regions correspond to small mean shifts, while low density regions correspond to large mean shifts. In this way, the shifts are always in the direction of the mode, i.e., the probability density maximum. The mean shift algorithm can be used as a tool for any feature space analysis, and an outline of a general procedure is as follows:
(1) The image domain is mapped into the feature space.
(2) An adequate number of search windows are defined at random locations in the space.
(3) The high density region centers are found by applying the mean shift algorithm to each window.
(4) The extracted centers are validated with image domain constraints in order to provide the feature palette.
(5) All of the feature vectors are allocated to the feature palette using image domain information

Automatic image segmentation is thus obtained following the guidelines described above and is presented in full detail in the specific case of image segmentation in (Comaniciu and Meer, 1997). This technique is dependent on a minimal number of parameters. The first parameter is the most general parameter that characterizes a segmentation technique: segmentation resolution. The algorithm as implemented in (Comaniciu and Meer, 1997) distinguishes segmentation resolution into three important classes: undersegmentation corresponds to the lowest resolution in which the region boundaries are the dominant edges in the image; oversegmentation corresponds to intermediate resolution in which the image is broken into many small regions; quantization corresponds to the highest resolution which contains all of the important colors. The second and last parameter is the maximum number of colors (or gray tones in the case of a gray scale image) the image can be classified into. This parameter can also be thought of as the maximum number of classes that the MSC algorithm can distinguish. In our particular case, we chose to use the under-segmentation class since we are interested in finding the dominant edges in the image (i.e., the LI and MA profiles), and we chose an initial maximum number of gray tones equal to three. This value was specifically chosen since the goal is to classify three different regions of the considered guidance zone: the adventitia layer, the intima and media layers, and the lumen. As defined in (Comaniciu and Meer, 1997), the under-segmentation class is then translated into three parameters used in the mean shift algorithm:
(1) the radius of the search window, r, is taken to be equal to 4, defined as the square root of the global covariance matrix;
(2) the smallest number of elements that are required for a significant color, Nₘᵢₙ, is taken to be equal to 100;
(3) the smallest number of contiguous pixels required for a significant image region, N_{con}, is taken to be equal to 10.

An example output image from the mean shift algorithm is displayed in FIG. 15 as colored overlay on the original grayscale Guidance Zone.

Ideally, the borders between the three found classes should represent the desired LI and MA profiles, but as FIG. 15 clearly shows, this is often not the case. In fact, ultrasound images are typically noisy images and can be affected by backscattering in the lumen. Another factor that prevents the use of this ideal solution (i.e., using the raw borders between the classes as the LI and MA profiles) is that the image was cropped so as to contain the entire guidance zone in a rectangle, and in many cases contains sections of the image that are found below the AD_{F} profile and/or farther above in the lumen. This problem is amplified in the case of inclined or curved arteries, since the rectangle containing the entire guidance zone assumes a more extended height and hence contains more tissue structures not found in the original guidance zone. These problems can lead to a jagged border between two classes, the presence of numerous small sections of one class enclosed inside a larger section of another class and/or a border that does not correctly run along the true LI and MA borders. It is therefore evident that a refinement process must be implemented before the final LI and MA borders can be defined.

### LI/MA Refinement Process:

The first challenge in the refinement process is to automatically find the correct class that corresponds to the intima and media layers (IM_{class}). First of all, the class which contains the adventitia layer (AD_{F}_{_{class}}) is found by determining which classes correspond to each point of the AD_{F} profile, and then establishing the AD_{F}_{_{class}} as the most recurring class. Subsequently, the output image is examined column by column starting from the AD_{F} point and the class found at the first transition point is taken as the IM_{class} for that column. The final IM_{class} is then ascertained by finding the most recurring class. FIG. 15 reports the segmented IM_{class} as a binary (i.e., black and white) mask. FIG. 13 shows the reconstruction of the MA profile from the MSC. It has five parts: A, B, C, D, E and F. Part A portray the output class image from the MSC algorithm with a sample profile (i.e., column) highlighted in the image. Part B shows the intensity profile of such profile, demonstrating the process. Part C shows the binary image is subsequently created in which the objects correspond to the pixels of the image that were classified as belonging to the IM_{class}.

A preliminary step of the refinement process is to remove all objects that are not included in the original guidance zone. This is useful so as to be sure that only sections of the image contained in the original region of interest are considered, and thus removes the problem of classifying unwanted tissue lying beneath the AD_{F} profile or too high above in the lumen (FIG. 16). After this step, exceedingly small objects (defined as those objects in the binary image which have an area (i.e., total number of connected pixels) less than [symbol] are eliminated (FIG. 17). Our experimental data showed that [symbol] =7 is an optimal value to guarantee the removal of small objects that may remain after those not included in the guidance zone are eliminated, but to not discard relatively small objects of interest that are fundamental for establishing a correct MA or LI profile. A preliminary MA profile is then established by doing a column by column search for the first transition point from 0 to 1 found above the AD_{F} profile point for the considered column. This finds the first point belonging to the IM_{class} avoiding however including pixels that are found directly above the AD_{F} profile point. Once this profile is found, the following steps are implemented to: determine the final MA profile:
(1) Divide the preliminary profile into different trends. Considering profile_{y} as the row value of a certain point in the preliminary profile, and profile_{y+1} as the row value of its contiguous point, the two points can be considered as belonging to the same trend if the following equation is respected:

|profile_{y}−profile_{y+1}|≦δ (1)
So, two contiguous points that have a vertical distance greater than δ are classified as belonging to two separate trends. This threshold was chosen to be equal to 3 pixels which correspond roughly to 0.2 mm, which is about one forth the value of a normal IMT. This value separates the profile into trends that are irregular but ensures the possibility of not distinguishing too many trends in the case of a curved vessel.
(2) Initialize the profile as the longest trend that is found (FIG. 18 or Part E of the FIG. 13).
(3) Examine the remaining trends one by one and are classify them as belonging to the profile if two conditions are met. For the sake of clarity, let A and B be the two closest points of the separate trends.

|A_{y}−B_{y}|≦φ (2)

|Aₓ−Bₓ|≦φ (3)
The first check is to help avoid connecting a trend to the profile which is found too far above, while the second check is used to avoid connecting two trends that are too far apart from one another, which could cause errors in the final determination of the profile, especially in the case of inclined or curved vessels. Pilot studies we performed on our image dataset demonstrated that φ taken to be equal to 4 if the two points are contiguous or equal to 7 in any other case are suitable thresholds for linking two separate trends. 7 pixels approximately corresponds to 0.45 mm which is half of the normal IMT value and permits linking trends that are not right next to each other and perfectly aligned. The lower value in the case of trends that are next to each other was chosen to allow linking in the case of curved or inclined arteries but to preclude linking of a trend that is too far from the profile. Our studies showed instead that a suitable value for φ is equal to 20 pixels.
(4) B-spline fit the different trends to provide the final profile (FIG. 18, Part F of the FIG. 13).
   The LI border that is subsequently established is strongly dependent on the computed MA border, since the preliminary LI profile is found by doing a column by column search starting from the MA point of the considered column. Therefore, only the columns where a MA point was found are considered. The LI point for each column is determined as the first transition point from 1 to 0. A check is done on the distance between the MA point and the found LI point to be sure to skip over potential small holes found in the binary image which are too near to the MA border.

The final LI profile is then ascertained by following the same 4 steps described in the previous subsection for the determination of the MA profile (FIG. 20 and FIG. 21).

### Final Refinement Checks.

Once the two borders of interest are computed, two final checks are carried out. The first check, by anatomic (lumen) reference, is to avoid the potential error case in which the computed LI profile falls inside the lumen area. This error can be due to the fact that the intima and media layers acquire a dark aspect in the ultrasound image and therefore the MSC classifier correctly identifies the two classes whose border gives the MA profile but associates the pixels belonging to the artery wall as part of the lumen, and therefore does not correctly identify the LI border. So to prevent this, the same lumen validation process as described herein is used. The computed LI profile is therefore defined as falling inside the lumen area if more than 25% of the LI points are classified as belonging to the lumen area of the vessel as determined by the support routine. If this should be the case, the MSC algorithm is rerun on the image, but this time using a maximum number of classes N_{colors} equal to 4 (compared to the original maximum number, 3) and reducing the guidance zone. Thus we call this approach as recursive approach to LI/MA estimation. Since the dark aspect of the intima and media layers may also polarize the performance of the lumen validation process, the reduced guidance zone is initialized as solely the portion of the image that is included in the original guidance zone and that is not classified as the lumen area by the support routine. Then a column by column search is done to verify that the upper limit of the new guidance zone of the considered column is found at least [symbol] pixels above the MA point for the same column. If this is not verified, the upper limit for such column is empirically imposed to be [symbol] pixels above the MA point, ensuring therefore that it encloses the vessel wall. We took [symbol] to be equal to 15, and this value was considered optimal after pilot studies on our database. The second check, by relative IMT measurement reference, is to avoid the potential error case in which the MSC classifier mistakenly associates a large section of the intima and media layer as belonging to the adventitia layer. This causes a computed MA profile which incorrectly lies in between the true. MA and LI borders. To remedy this, the following distances are calculated:

D₁=PD(MA,AD_{F}) (4)

D₂=PD(LI,AD_{F}) (5)

The ratio

$$\frac{{D}_{1}}{{D}_{2}}$$

is subsequently calculated, which gives a relative measure of the IMT value, therefore avoiding imposing an absolute cut-off value which could prove troublesome in the case of different image resolutions. So, if the computed MA border is found in between the two border walls, the calculated ratio increases with respect to a correct case since D1 is higher. Pilot studies performed on our image database showed that, even in the case of irregular AD_{F} profiles, images in which the LI and MA borders were correctly identified produced a

$$\frac{{D}_{1}}{{D}_{2}}$$

ratio lower ratio lower than [symbol] . This parameter [symbol] was taken as a cut-off limit for this check and was equal to 0.6. If an image does not pass this test, the MSC algorithm is rerun on the image, this time using N_{colors} equal to 4 while still keeping the original guidance zone size. As described above, both of these cases for which a final refinement check is required are due to an incorrect initial classification, and our method for resolving the problem consists in raising the number of classes which the mean shift classifier can distinguish. This extra class allows the classifier to ‘distinguish’ the two separate classes that originally were merged together in one. The final LI and MA borders are represented by FIG. 22.

### Performance Metric:

We tested CAILRS was tested on a multi-institutional database consisting of 300 longitudinal B-mode ultrasound images of the common carotid artery. Different experts then manually traced the LI and MA profiles in all of the images. The manual profiles were consequently interpolated by a B-spline and averaged. The averaged profile was considered as ground truth (GT). The performance of the automatic tracings of the LI and MA profiles was then assessed by calculating the overall system distance of the LI/MA traced profiles from the GT profiles and of the IMT measurement bias. We adopted the Polyline distance (PD) as proposed by Suri et al in 2000 (Suri, 2000) for all distance calculations. These distance metric measures the distance between each vertex of a boundary and the segments of the other boundary. We chose to use this distance metric because it appears to be a robust and reliable indicator of the distance between two boundaries and does not depend on the number of points in either boundary. So the overall system error of the LI/MA traced profiles from the GT profiles are calculated as:

ε_{GTLI}^{AutoLI}=PD(CAILRS_{LI},GT_{LI}) (6)

ε_{GTMA}^{AutoMA}=PD(CAILRS_{MA},GT_{MA}) (7)

whereas the IMT measurement bias ε_{CAILRS}^{IMT} is found by computing first the IMT using the mean shift method and comparing with the IMT using the GT borders:

IMT_{CAILRS}=PD(CAILRS_{LI},CAILRS_{MA}) (8)

IMT_{GT}=PD(GT_{LI},GT_{MA}) (9)

ε_{CAILRS}^{IMT}=IMT_{CAILRS}−IMT_{GT} (10)

The IMT bias is intentionally calculated without an absolute value in order to give an idea of how much the algorithm underestimates and/or overestimates the IMT measure. The PD distance is initially calculated in pixels, but this distance is then converted into millimeters for the final performance evaluation. The conversion was carried out thanks to a calibration factor which is equal to the axial spatial resolution of the images. The values of these calibration factors for the images deriving from the different institutions was discussed above. For an overall assessment of the algorithm performance, the Figure of Merit (FoM) was calculated, and is defined by the following formula:

$$FoM = ( 1 - \frac{\vert {IMT}_{GT} - {IMT}_{CAILRS} \vert}{{IMT}_{GT}} ) \times 100 \qquad ( 11 )$$

### Performance Evaluation

The average ε_{GTLI}^{AutoLI} is equal to 0.5036 mm while the standard deviation is equal to 0.9952 mm. The average ε_{GTMA}^{AutoMA}, instead, is equal to 0.4180 mm while the standard deviation is equal to 0.8718 mm. The mean ε_{CAILRS}^{IMT} is equal to −0.069 mm while the standard deviation is equal to 0.299 mm. This distribution suggests that the AutoLI and AutoMA tracings can be clinically used for IMT computation. The overall FoM was found to be equal to 94.9%, and we found that has a slight tendency towards underestimation of the true IMT measurement. Table 3 shows the performance of CAILRS with respect to already published methods.

Benchmark with a Semi-Automatic Technique:

To provide a more complete overview of the performances of our CAILRS automated algorithm, we benchmarked the results with a semi-automatic technique used for IMT measurement. This technique automatically extracts the LI and MA profiles from a region of interest containing the distal wall. It however requires a human operator to first manually select this ROI, precluding complete automation. This algorithm is based on a first-order absolute moment edge operator (FOAM) and can be found in full detail in (Faita et al., J Ultrasound Med 2008; 27:1353-61). Both of the considered algorithms were tested on the same image database. The first two rows of Table 3 summarizes the performance of CAILRS and FOAM. FOAM presents a ε_{GTLI}^{LI} value equal to 0.3810±0.9603 mm while ε_{GTMA}^{MA} is equal to 0.5168±0.9220 mm.

The overall FoM for FOAM was found to be equal to 98.0%. FOAM also tends to underestimate the IMT value. Due to the fact that each algorithm was not able to process all of the images, the Ground Truth IMT average values shown in the fifth column of the table are slightly different.

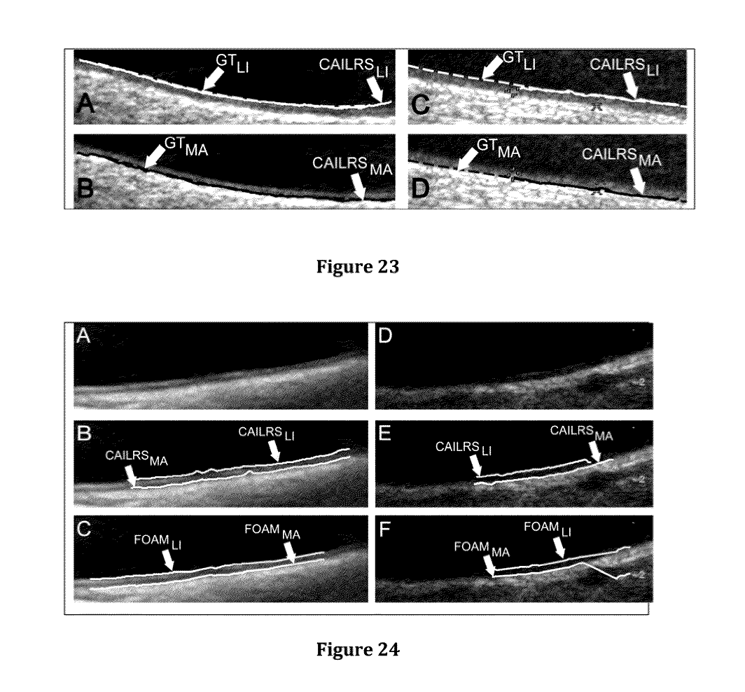

FIG. 23 shows a comparison of the CAILRS and FOAM results. The first row shows the original automatically cropped image; the second row shows the computed CAILRS LI and MA tracings; the bottom row shows the computed FOAM LI and MA borders. The first column shows an image where both techniques accurately trace the two borders, while the second column portrays a slightly inclined artery wall, which presents problems for the FOAM algorithm, as can be seen in FIG. 24.F. This is due to the fact that the ROI selected by the human operator contains tissue lying underneath the adventitia layer. This problem is remedied in our new CAILRS algorithm thanks to the automatic tracing of the adventitia layer.

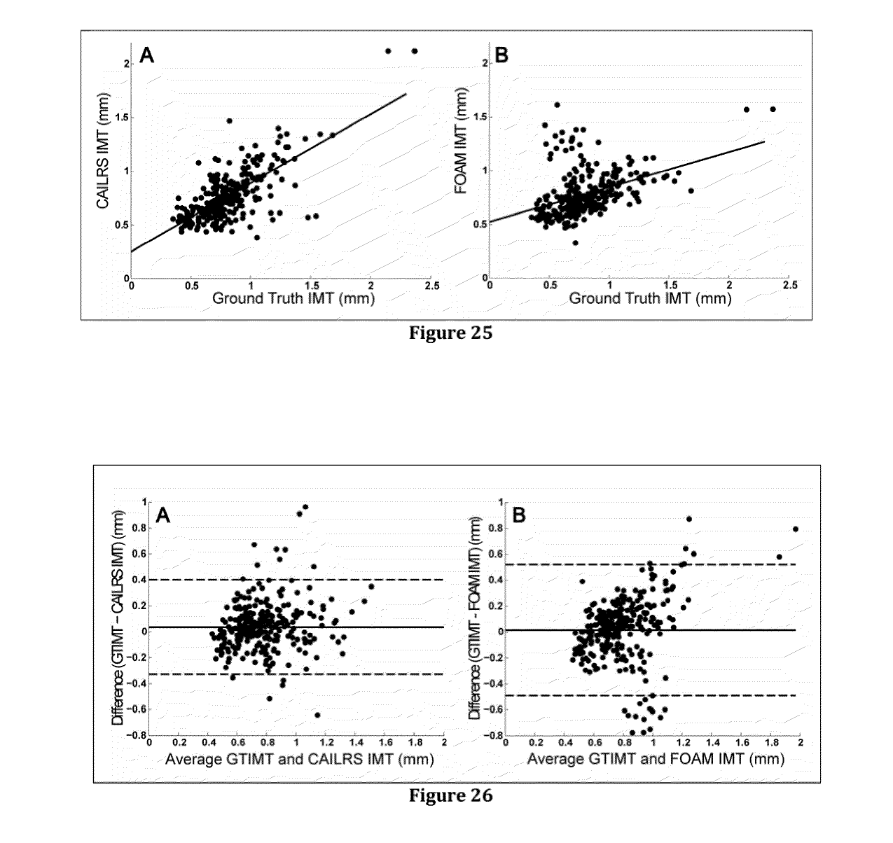

FIG. 25 and FIG. 26 shows the correlation plot and the Bland-Altman plot of the IMT biases of the two techniques.

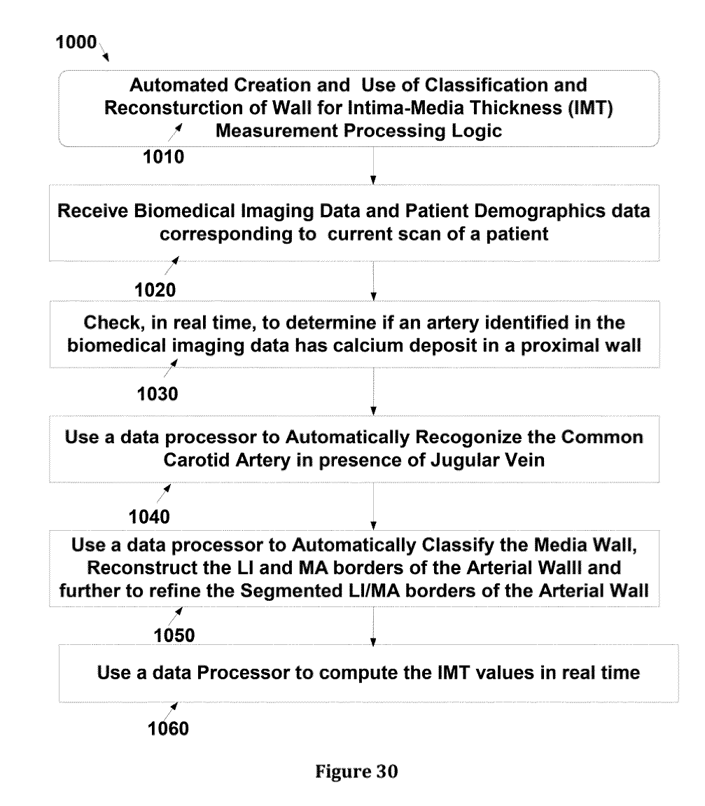

FIG. 30 is a processing flow diagram illustrating an example embodiment of an automated IMT measurement system in carotid, brachial, aorta and femoral arteries herein (block 1000). The method of an example embodiment includes: receiving biomedical imaging data corresponding to a current scan of a patient (processing block 1020); checking the calcium content in the ultrasound image (processing block 1030); automated recognition of artery in the presence of artery and jugular vein (processing block 1040); automated classification of media wall, LI/MA reconstruction, and refinement (processing block 1050); and IMT measurement (processing block 1060).

The evaluation of the carotid artery wall is essential for the assessment of a patient's cardiovascular risk or for the diagnosis of cardiovascular pathologies. This patent application presents a new completely user-independent algorithm called CAILRS, which automatically segments the intima layer of the far wall of carotid ultrasound artery based on recursive mean shift classification applied to the far wall. Further, the system extracts the lumen-intima and media-adventitia borders in the far wall of the carotid artery. The CAILRS system is characterized and validated by comparing CAILRS borders with the manual tracings carried out by experts. The new technique is also benchmarked with a semi-automatic technique based on a first-order absolute moment edge operator (FOAM) and compared to our previous edge-based automated methods such as: CALEX (J Ultras Med 2010a, 29:399-418), CULEX (IEEE Transactions on Instrumentation and Measurement, 2007; 56:1265-74), CAUDLES (Molinari F, Meiburger K M, Zeng G, Nicolaides A, Suri JS, CAUDLES-E F: Carotid Automated Ultrasound Double Line Extraction System Using Edge Flow. J Digit Imaging, 2011), and CALSFOAM (Molinari F, Liboni W, Pantziaris M, Suri J S, “CALSFOAM—Completed Automated Local Statistics based first order absolute moment” for carotid wall recognition, segmentation and IMT measurement: validation and benchmarking on a 300 patient database. International Angiology, 2011). In this application, we used 300 longitudinal B-mode carotid images. In comparison to semi-automated FOAM, CAILRS showed the IMT bias of −0.035±0.186 mm while FOAM showed as −0.016±0.258 mm. Our IMT was slightly underestimated with respect to the ground truth IMT, but showed a uniform behavior over the entire database. CAILRS outperformed all the four previous automated methods. The system's Figure of Merit (FoM) was 95.4%, which was lower than that of the semi-automated method (98%), but higher than that of the other automated techniques.

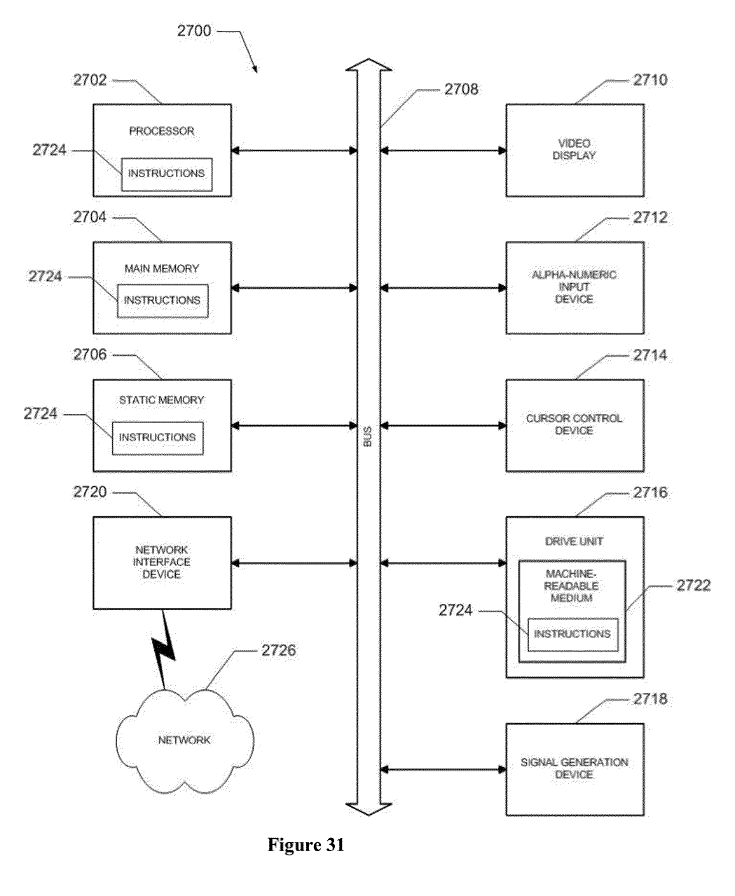

FIG. 31 shows a diagrammatic representation of machine in the example form of a computer system 2700 within which a set of instructions when executed may cause the machine to perform any one or more of the methodologies discussed herein. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, iPad, iPhone or hand held tablet, a web appliance, a network router, switch or bridge, or any machine capable of executing a set of instructions (sequential or otherwise) that specify actions to be taken by that machine. Further, while only a single machine is illustrated, the term “machine” can also be taken to include any collection of machines that individually or jointly execute a set (or multiple sets) of instructions to perform any one or more of the methodologies discussed herein.

The example computer system 2700 includes a processor 2702 (e.g., a central processing unit (CPU), a graphics processing unit (GPU), or both), a main memory 2704 and a static memory 2706, which communicate with each other via, a bus 2708. The computer system 2700 may further include a video display unit 2710 (e.g., a liquid crystal display (LCD) or a cathode ray tube (CRT)). The computer system 2700 also includes an input device 2712 (e.g., a keyboard), a cursor control device 2714 (e.g., a mouse), a disk drive unit 2716, a signal generation device 2718 (e.g., a speaker) and a network interface device 2720.

The disk drive unit 2716 includes a machine-readable medium 2722 on which is stored one or more sets of instructions (e.g., software 2724) embodying any one or more of the methodologies or functions described herein. The instructions 2724 may also reside, completely or at least partially, within the main memory 2704, the static memory 2706, and/or within the processor 2702 during execution thereof by the computer system 2700. The main memory 2704 and the processor 2702 also may constitute machine-readable media. The instructions 2724 may further be transmitted or received over a network 2726 via the network interface device 2720. While the machine-readable medium 2722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any non-transitory medium that is capable of storing, encoding or carrying a set of instructions for execution by the machine and that cause the machine to perform any one or more of the methodologies of the various embodiments, or that is capable of storing, encoding or carrying data structures utilized by or associated with such a set of instructions. The term “machine-readable medium” can accordingly be taken to include, but not be limited to, solid-state memories, optical media, and magnetic media.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing. Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims, reflect, inventive subject matter lies in less than all features of a single disclosed embodiment. Thus the following claims are hereby incorporated into the Detailed Description, with each claim standing on its own as a separate embodiment.

What is claimed is:

1. A computer-implemented method comprising:
receiving biomedical imaging, data and patient demographic data corresponding to a current scan of a patient's carotid, brachial, femoral and arotic vessels;
real time checking if the artery has calcium deposit or no calcium deposit in the proximal wall;
acquiring the arterial data as a combination of longitudinal B-mode and transverse B-mode or longitudinal B-mode alone;
using a data processor to automatically recognize the artery by embedding anatomic information;
using the data processor to calibrate the region of interest around the automatically recognized artery using recursive regional information such as a pixel-based classifier; and determining the IMT of the arterial wall.

2. The method as claimed in claim 1 wherein the biomedical imaging data comprises of combination of two-dimensional (2D) longitudinal B-mode and two-dimensional (2D) transverse B-mode ultrasound images or longitudinal B-mode alone, with and without the presence of the calcium and applicable to Carotid, Brachial, Femoral and Aorta Vessels.

3. The method as claimed in claim 1 wherein the method can be for automated recognition using a multi-resolution approach, where the edges of the MA border are determined in coarse resolution and applicable to applicable to Carotid, Brachial, Femoral and Aorta Vessels.

4. The method as claimed in claim 1 wherein the method can be for automated recognition using a multi-resolution approach, where the edges of the MA border are determined in coarse resolution and up-sampled back onto the original high resolution image. This is applicable to applicable to Carotid, Brachial, Femoral and Aorta. Vessels.

5. The method as claimed in claim 1 wherein the method can be for automated recognition using a multi-resolution approach, and the artery location can be validated using anatomic reference information such as lumen and applicable to Carotid, Brachial, Femoral and Aorta Vessels.

6. The method as claimed in claim 1 wherein the method can be for automated recognition using a multi-resolution approach, and the artery location can be validated using anatomic information such as lumen. The lumen is automatically located in the image frame using the statistical classifier and applicable to Carotid, Brachial, Femoral and Aorta Vessels.

7. The method as claimed in claim 1 wherein the method where the calibration stage is guided by the automated recognition of the artery, which has been validated by the anatomic information, which in turn is computed automatically using a statistical classifier and applicable to Carotid, Brachial, Femoral and Aorta Vessels.

8. A computer-implemented method comprising:
receiving biomedical imaging data and patient demographic data corresponding to a current scan of a patient's carotid, brachial, femoral and arotic vessels;
real time checking if the artery has calcium deposit in the proximal wall;
acquiring the arterial data as a combination of longitudinal B-mode and transverse B-mode;
using a data processor to automatically recognize the artery;
using the data processor to calibrate the region of interest around the automatically recognized artery; and determining the IMT of the arterial wall; where,
the automated recognition is computed in coarse resolution by convolution of higher order derivative of Gaussian kernels, the scale of the kernel is computed using a priori anatomic information from the database of ultrasound images.

9. The method as claimed in claim 10 wherein the method where the automated recognition using higher order derivative can be with and without calcium present in the arterial proximal wall.

10. The method as claimed in claim 10 wherein the method where the automated recognition can be a feature based method unlike the multi-resolution higher order derivative approach. This automated recognition method guides the stage-H (calibration method) for regional classification and MA/LI reconstruction.

11. The method as claimed in claim 10 wherein the method where calibration (stage II) which is a classifier based on mean shift using either a three classes (adventitia class, media class and the background class) or a four classes (adventitia class, media class-A, media-class B and background class).

12. The method as claimed in claim 10 wherein the method where calibration which is classifier based on mean shift using either a three classes or a four classes. The choice of the MA border detection and reconstruction is based on media class and transition from adventitia class to media class in the binary image by thresholding the MSC classified image.

13. The method as claimed in claim 10 wherein different trends of MA borders computed and connected to reconstruct the MA border. These trends (broken MA segments) are checked for connectivity by looking at the profile differences |profile_{y}−profile_{y+1}|≦δ and two conditions: |A_{y}−B_{y}|≦φ and |Aₓ−Bₓ|≦φ, where delta and phi are connectivity thresholds.

14. The method as claimed in claim 10 wherein the LI border is reconstructed from reference of MA border. The LI border is reconstructed in the same way MA border. The LI border is checked for lumen penetration and is corrected by readjusting the number of classes of MSC from three to four and re-running the MA and LI border again (recursively).

15. The method as claimed in claim 10 wherein the LI border is reconstructed from reference of MA border. The LI border is reconstructed in the same way MA border. The LI border is checked for lumen penetration and is corrected by readjusting the number of classes of MSC from three to four and re-running the MA and LI border again (recursively). This lumen is identified using a statistical classifier, just like the automated recognition (ADF profile) is validated using the statistical classifier.

16. The method as claimed in claim 10 wherein the LI and MA borders are refined and checked with respect to the ADF profile computed from the stage-I. This refinement check is done by computing the ratio of D1 to D2, where D₁=PD(MA,AD_{F}) and D₂=PD(LI, AD_{F}). D1 is the distance between MA and ADF profile while D2 is the distance between LI and ADF profile. LI and MA borders are recomputed with higher number of MSC classes if ratio
$$\frac{{D}_{1}}{{D}_{2}}$$
is then thresh is above a threshold (computed empirically).

17. LI border is reconstructed from reference of MA border. The LI border is reconstructed in the same way MA border. The LI border is checked for lumen penetration and is corrected by readjusting the number of classes of MSC from three to four and re-running the MA and LI border again (recursively). This lumen is identified using a statistical classifier, just like the automated recognition (ADF profile) is validated using the statistical classifier.

18. The method as claimed in claim 1 wherein the method can be used for monitoring the IMT for patients, taking the IMT over time and tracking the IMT values during the follow-up studies. The method is also used for computing the IMT of the batch of patients in clinical databases automatically using the knowledge of ethnicity, demographics, age and gender and super controlled by the user.

19. The method as claimed in claim 1 wherein the method where calibration can be changed from mean shift classifier, to a deformable model, to an edge detector, by a combination of an edge detector and a deformable model.

20. The method as claimed in claim 1 wherein the method is can be run on a iPad, iPhone or running on a tablet using. Android Operating System (called AtheroMobile™). The IMT-based implementation is using Java wrapper and the Graphical User Interface and can be used by a vascular surgeon or trained sonographers or vascular radiologist, neuroradiologist or even a cardiologist.
